(19) United States
(12) Patent Application Publication
(10) Pub. No.: US 2012/0190825 A1
(43) Pub. Date: Jul. 26, 2012

(54) ACETYL LYSINE INCORPORATION WITH tRNA SYNTHETASE

(75) Inventors: Heinz Neumann, Cambridge (GB); Jason Chin, Cambridge (GB)

(73) Assignee: MEDICAL RESEARCH COUNCIL, London (GB)

(21) Appl. No.: 13/499,340

(22) PCT Filed: Oct. 1, 2010
(86) PCT No.: PCT/GB2010/001848

§ 371 Date: Mar. 30, 2012

(30) Foreign Application Priority Data
Oct. 1, 2009 (GB) 0917242.0

(51) Int. Cl. C12P 21/02; C12N 1/21; C07K 14/435; C12N 15/63; C12N 9/00; C12N 15/52

(52) U.S. Cl. 530/358; 435/183; 536/23.2; 435/68.1; 435/320.1; 435/252.33

(57) ABSTRACT

The invention relates to a tRNA synthetase capable of binding Nε-acetyl lysine, wherein said synthetase comprises a polypeptide having at least 90% sequence identity to the amino acid sequence of MbPyIRS, and wherein said synthetase comprises a L266M mutation.

### FIELD OF THE INVENTION

The invention is in the field of production of biologically important macromolecules which are acetylated. In particular, the invention is in the field of incorporation of N^{ε}-acetyl-lysine into polypeptides.

### BACKGROUND TO THE INVENTION

The genetic code of prokaryotic and eukaryotic organisms has been expanded to allow the in vivo, site-specific incorporation of over 20 designer unnatural amino acids in response to the amber stop codon. This synthetic genetic code expansion is accomplished by endowing organisms with evolved orthogonal aminoacyl-tRNA synthetase/tRNA_{CUA} pairs that direct the site-specific incorporation of an unnatural amino acid in response to an amber codon. The orthogonal aminoacyl-tRNA synthetase aminoacylates a cognate orthogonal tRNA, but no other cellular tRNAs, with an unnatural amino acid, and the orthogonal tRNA is a substrate for the orthogonal synthetase but is not substantially aminoacylated by any endogenous aminoacyl-tRNA synthetase.

Genetic code expansion in E. coli using evolved variants of the orthogonal Methanococcus jannaschii tyrosyl-tRNA synthetase/tRNA_{CUA} pair greatly increases unnatural amino acid-containing protein yield since, in contrast to methods that rely on the addition of stoichiometrically pre-aminoacylated suppressor tRNAs to cells or to in vitro translation reactions, the orthogonal tRNA_{CUA} is catalytically re-acylated by its cognate aminoacyl-tRNA synthetase enzyme, thus aminoacylation need not limit translational efficiency.

Many potential applications of unnatural amino acid mutagenesis, including the translational incorporation of amino acids corresponding to post-translational modifications present at multiple sites in proteins such as acetylation, require more efficient methods of incorporation to make useful amounts of protein. Moreover the introduction of biophysical probes and chemically precise perturbations into proteins in their native cellular context offers the exciting possibility of understanding and controlling cellular functions in ways not previously possible.

N^{ε}-acetylation of lysine is a reversible post-translational modification with a regulatory role to rival phosphorylation in eukaryotic cells¹⁻¹⁴. No general methods to synthesize proteins containing N^{ε}-acetyl-lysine at defined sites exist.

N^{ε}-acetylation of lysine was first described on histones²¹. Lysine acetylation and de-acetylation are mediated by histone acetyl transferases (HATs) and histone deacetylases (HDACs) respectively. In recent years it has emerged that hundreds of eukaryotic proteins (beyond histones) are regulated by acetylation, including more than 20% of mitochondrial proteins²⁰.

Despite the huge importance of lysine acetylation there is no general method of producing homogeneous recombinant proteins that contain N^{ε}-acetyl-lysine at defined sites. Semi-synthetic methods to install N^{ε}-acetyl-lysine using native chemical ligation were employed in demonstrating the role of H4 K16 in chromatin decompaction¹. These studies give a taste of the impact that a general method to produce homogeneously acetylated proteins would have on our understanding of the molecular role of acetylation in biology.

Current chemical based methods of acetylation require the synthesis of large quantities of modified peptide thioester, which is a drawback. Furthermore, such known methods suffer from limitation to N-terminal residues.

Some researchers have used purified HAT complexes to acetylate recombinant proteins. However this is often an unsatisfactory solution because: i) the HATs for a particular modifications may be unknown; ii) tour-de-force efforts are often required to prepare active HAT complexes; iii) HAT mediated reactions are often difficult to drive to completion leading to a heterogeneous sample; and iv) HATs may acetylate several sites, making it difficult to interrogate the molecular consequences of acetylation at any one site.

WO2009/056803 discloses a way of exploiting the naturally occurring polypeptide synthesis machinery (translational machinery) of the cell in order to reliably incorporate Nε-acetyl lysine into polypeptides at precisely defined locations. Specifically, the document discloses a tRNA synthetase which has been modified to accept Nε-acetyl lysine and to catalyse its incorporation into transfer RNA (tRNA). This novel enzyme was evolved into a suitable tRNA synthetase/tRNA pairing which could be used in order to specifically incorporate Nε-acetyl lysine into proteins at the point of synthesis and at position(s) chosen by the operator.

The present invention seeks to overcome problem(s) associated with the prior art.

### SUMMARY OF THE INVENTION

The present inventors provide for the first time a novel tRNA synthetase, and a corresponding new approach to the production of polypeptides incorporating N^{ε}-acetyl lysine. These new materials and techniques enable the production of homeogeneous samples of polypeptide which each comprise the desired post translational modification.

The key difference from prior art synthetases is the L266M mutation i.e. the synthetase suitably comprises methionine at the position corresponding to amino acid L266 of the wild type sequence. This specific mutation has not been taught before. The advantage of the invention is superior efficiency of acetyl lysine incorporation compared to prior art techniques. These effects are demonstrated in the examples section by direct comparison to the less efficient prior art synthetases.

The invention is based upon these remarkable findings.

Thus in one aspect the invention provides a tRNA synthetase capable of binding N^{ε}-acetyl lysine, wherein said synthetase comprises a polypeptide having at least 90% sequence identity to the amino acid sequence of MbPylRS, and wherein said synthetase comprises a L266M mutation.

In another aspect, the invention relates to a tRNA synthetase as described above wherein said tRNA synthetase comprises amino acid sequence corresponding to the amino acid sequence of at least L266 to C313 of MbPylRS, or a sequence having at least 90% identity thereto.

In another aspect, the invention relates to a tRNA synthetase as described above wherein said polypeptide comprises a mutation relative to the wild type MbPylRS sequence at one or more of L270, Y271, L274 or C313.

In another aspect, the invention relates to a tRNA synthetase as described above wherein said at least one mutation is at L270, L274 or C313.

In another aspect, the invention relates to a tRNA synthetase as described above which comprises Y271F.

In another aspect, the invention relates to a tRNA synthetase as described above which comprises L2701, Y271F, L274A, and C313F.

In another aspect, the invention relates to a nucleic acid comprising nucleotide sequence encoding a polypeptide as described above.

In another aspect, the invention relates to use of a polypeptide as described above to charge a tRNA with N^{ε}-acetyl lysine. Suitably said tRNA comprises MbtRNA_{CUA}. Suitably said tRNA comprises MbtRNA_{CUA} (i.e. suitably said tRNA comprises the publicly available wild type Methanosarcina barkeri tRNACUA sequence as encoded by the MbPylT gene).

In another aspect, the invention relates to a method of making a polypeptide comprising N^{ε}-acetyl lysine comprising arranging for the translation of a RNA encoding said polypeptide, wherein said RNA comprises an amber codon, wherein said translation is carried out in the presence of a polypeptide as described above and in the presence of tRNA which recognises the amber codon and is capable of being charged with N^{ε}-acetyl lysine, and in the presence of N^{ε}-acetyl lysine.

In another aspect, the invention relates to a method as described above wherein said translation is carried out in the presence of an inhibitor of deacetylation.

In another aspect, the invention relates to a method as described above wherein said inhibitor comprises nicotinamide (NAM).

In another aspect, the invention relates to a method as described above wherein said polypeptide comprises a histone protein.

Suitably the histone comprises a histone selected from H2A, H2B and H3.

### Suitably Either

(a) the histone is H3 and the lysine residue is lysine 56; or

(b) the histone is H2A and the lysine residue is lysine 9; or

(c) the histone is H2B and the lysine residue is lysine 5 and/or lysine 20.

In another aspect, the invention relates to use of a histone protein as described above in monitoring DNA breathing.

In another aspect, the invention relates to a homogenous recombinant histone, wherein said protein is made by a method as described above.

A vector comprising nucleic acid as described above.

Suitably vector further comprises nucleic acid sequence encoding a tRNA substrate of said tRNA synthetase.

Suitably said tRNA substrate is encoded by the MbPylT gene. Suitably said vector further comprises nucleic acid sequence encoding a tRNA substrate of said tRNA synthetase. Suitably said tRNA substrate is encoded by the MbPylT gene (see above).

In another aspect, the invention relates to a cell comprising a nucleic acid as described above, or comprising a vector as described above.

In another aspect, the invention relates to a cell as described above which further comprises an inactivated de-acetylase gene.

In another aspect, the invention relates to a cell as described above wherein said deactivated de-acetylase gene comprises a deletion or disruption of CobB.

### DETAILED DESCRIPTION OF THE INVENTION

To address the prior art deficit in methods to synthesize acetylated proteins we envisioned genetically encoding the incorporation of N^{ε}-acetyl-lysine into proteins with high translational fidelity and efficiency in response to the amber codon, via the generation of an orthogonal N^{ε}-acetyl-lysyl-tRNA synthetase/tRNA pair. Here we describe methods and materials for genetically incorporating N^{ε}-acetyl-lysine in response to the amber codon in Escherichia coli (E. coli), to produce site-specifically acetylated recombinant proteins. We further enable such proteins to be produced homogeneously, which has not been possible with prior art based techniques. We demonstrate that the Methanosarcina barkeri pyrrolysyl-tRNA synthetase (MbPylRS)/MbtRNA_{CUA} pair¹⁵⁻¹⁹ is orthogonal in E. coli, and has a comparable efficiency to a previously reported useful pair. We evolve this pair for site-specific incorporation of N^{ε}-acetyl-lysine in response to the amber codon with high translational fidelity and efficiency. Furthermore, we successfully eradicate the initially observed post-translational deacetylation. These strategies find wide application in deciphering the role of acetylation in the epigenetic code proposed for chromatin modifications^{2, 3}, and in a broader understanding of the cellular role of N^{ε}-acetylation²⁰.

### DEFINITIONS

The term ‘comprises’ (comprise, comprising) should be understood to have its normal meaning in the art, i.e. that the stated feature or group of features is included, but that the term does not exclude any other stated feature or group of features from also being present.

Networks of molecular interactions in organisms have evolved through duplication of a progenitor gene followed by the acquisition of a novel function in the duplicated copy. Described herein are processes that artificially mimic the natural process to produce orthogonal molecules: that is, molecules that can process information in parallel with their progenitors without cross-talk between the progenitors and the duplicated molecules. Using these processes, it is now possible to tailor the evolutionary fates of a pair of duplicated molecules from amongst the many natural fates to give a predetermined relationship between the duplicated molecules and the progenitor molecules from which they are derived. This is exemplified herein by the generation of orthogonal tRNA synthetase-orthogonal tRNA pairs that can process information in parallel with wild-type tRNA synthetases and tRNAs but that do not engage in cross-talk between the wild-type and orthogonal molecules. In some embodiments the tRNA itself may retain its wild type sequence. In those embodiments, suitably said entity retaining its wild type sequence is used in a heterologous setting i.e. in a background or host cell different from its naturally occurring wild type host cell. In this way, the wild type entity may be orthogonal in a functional sense without needing to be structurally altered. Orthogonality and the accepted criteria for same are discussed in more detail below.

The Methanosarcina barkeri PylS gene encodes the MbPylRS tRNA synthetase protein. The Methanosarcina barkeri PylT gene encodes the MbtRNA_{CUA} tRNA.

There are two closely related known aminoacyl-tRNA synthetase sequences, which we designated AcKRS-1 and AcKRS-2 (see WO2009/056803). AcKRS-1 has five mutations (L266V, L270I, Y271F, L274A, C313F) while AcKRS-2 has four mutations (L270I, Y271L, L274A, C313F) with respect to MbPylRS. The synthetase sequences of the present invention are chartacterised by comprising the L266M mutation. Suitably a most preferred synthetase sequence of the present invention comprises L266M, L270I, Y271F, L274A, and C313F mutations; this sequence may be referred to as AcKRS-3.

### Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

In the context of the present document, a homologous amino acid sequence is taken to include an amino acid sequence which is at least 15, 20, 25, 30, 40, 50, 60, 70, 80 or 90% identical, preferably at least 95 or 98% identical at the amino acid level. Suitably this identity is assessed over at least 50 or 100, preferably 200, 300, or even more amino acids with the relevant polypeptide sequence(s) disclosed herein, most suitably with the full length progenitor (parent) tRNA synthetase sequence. Suitably, homology should be considered with respect to one or more of those regions of the sequence known to be essential for protein function rather than non-essential neighbouring sequences. This is especially important when considering homologous sequences from distantly related organisms.

Most suitably sequence identity should be judged across at least the contiguous region from L266 to C313 of the amino acid sequence of MbPylRS, or the corresponding region in an alternate tRNA synthetase.

The same considerations apply to nucleic acid nucleotide sequences, such as tRNA sequence(s).

### Reference Sequence

When particular amino acid residues are referred to using numeric addresses, the numbering is taken using MbPylRS (Methanosarcina barkeri pyrrolysyl-tRNA synthetase) amino acid sequence as the reference sequence (i.e. as encoded by the publicly available wild type Methanosarcina barkeri PylS gene Accession number Q46E77):

MDKKPLDVLI SATGLWMSRT GTLHKIKHYE VSRSKIYIEM
ACGDHLVVNN SRSCRTARAF RHHKYRKTCK RCRVSDEDIN
NFLTRSTEGK TSVKVKVVSA PKVKKAMPKS VSRAPKPLEN
PVSAKASTDT SRSVPSPAKS TPNSPVPTSA PAPSLTRSQL
DRVEALLSPE DKISLNIAKP FRELESELVT RRKNDFQRLY
TNDREDYLGK LERDITKFFV DRDFLEIKSP ILIPAEYVER
MGINNDTELS KQIFRVDKNL CLRPMLAPTL YNYLRKLDRI
LPDPIKIFEV GPCYRKESDG KEHLEEFTMV NFCQMGSGCT
RENLESLIKE FLDYLEIDFE IVGDSCMVYG DTLDIMHGDL
ELSSAVVGPV PLDREWGIDK PWIGAGFGLE RLLKVMHGFK
NIKRASRSES YYNGISTNL

This is to be used as is well understood in the art to locate the residue of interest. This is not always a strict counting exercise—attention must be paid to the context. For example, if the protein of interest is of a slightly different length, then location of the correct residue in that sequence corresponding to (for example) Y271 may require the sequences to be aligned and the equivalent or corresponding residue picked, rather than simply taking the 271^{st} residue of the sequence of interest. This is well within the ambit of the skilled reader.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably a randomisation of said site is used, for example as described herein in connection with the evolution and adaptation of tRNA synthetase of the invention. As a default mutation, alanine (A) may be used. Suitably the mutations used at particular site(s) are as set out herein.

Thus an L266M mutant is produced from the wild type sequence by changing L to M at the position corresponding to L266; using to illustrate this an L266M polypeptide would have the sequence:

MDKKPLDVLI SATGLWMSRT GTLHKIKHYE VSRSKIYIEM
ACGDHLVVNN SRSCRTARAF RHHKYRKTCK RCRVSDEDIN
NFLTRSTEGK TSVKVKVVSA PKVKKAMPKS VSRAPKPLEN
PVSAKASTDT SRSVPSPAKS TPNSPVPTSA PAPSLTRSQL
DRVEALLSPE DKISLNIAKP FRELESELVT RRKNDFQRLY
TNDREDYLGK LERDITKFFV DRDFLEIKSP ILIPAEYVER
MGINNDTELS KQIFRVDKNL CLRPMMAPTL YNYLRKLDRI
LPDPIKIFEV GPCYRKESDG KEHLEEFTMV NFCQMGSGCT
RENLESLIKE FLDYLEIDFE IVGDSCMVYG DTLDIMHGDL
ELSSAVVGPV PLDREWGIDK PWIGAGFGLE RLLKVMHGFK
NIKRASRSES YYNGISTNL

This applies equally to each of the other mutations discussed herein.

A fragment is suitably at least 10 amino acids in length, suitably at least 25 amino acids, suitably at least 50 amino acids, suitably at least 100 amino acids, suitably at least 200 amino acids, suitably at least 250 amino acids, suitably at least 300 amino acids, suitably at least 313 amino acids, or suitably the majority of the tRNA synthetase polypeptide of interest.

Suitably polypeptides of the invention are manufactured by causing expression of a nucleotide sequence encoding them, for example in a suitable host cell.

Nucleotide sequences of the invention are suitably those encoding the polypeptides of the invention.

An exemplary nucleotide sequence is produced by mutating the sequence encoding wild type Methanosarcina barkeri PylS polypeptide, which sequence is:

atggataaaaaaccattagatgttttaatatctgcgaccgggctctggatgtccaggact
ggcacgctccacaaaatcaaacactatgaggtctcaagaagtaaaatatacattgaaatg
gcgtgtggagaccatcttgttgtgaataattctaggagttgtagaacagccagagcattc
agacatcataagtacagaaaaacctgcaaacgatgtagggtttcggacgaggatatcaat
aatttcctcacaagatcaactgaaggcaaaaccagtgtgaaagttaaggtagtttctgct
ccaaaggtcaaaaaagctatgccgaaatcagtttcgagggctccaaagcctctggaaaat
cctgtgtctgcaaaggcatcaacggacacatccagatctgtaccttcgcctgcaaaatca
actccaaattcgcctgttcccacatcggctcctgctccttcacttacaagaagccagctc
gatagggttgaggctctcttaagtccagaggataaaatttctctgaatattgcaaagcct
ttcagggaacttgagtccgaacttgtgacaagaagaaaaaacgattttcagcggctctat
accaatgatagagaagactaccttggtaaactcgaacgggacattacgaaatttttcgta
gaccgggattttctggagataaagtctcctatccttattccggcagaatacgtggagaga
atgggtattaacaatgatactgaactttcaaaacagatcttcagggtggataaaaatctc
tgcttaaggccaatgcttgccccgactctttacaactatctgcgaaaactcgataggatt
ttaccagatcctataaagattttcgaagtcgggccctgttaccggaaagagtctgacggc
aaagagcacctggaagaatttaccatggtgaacttctgtcagatgggttcgggatgtact
cgggaaaatcttgaatccctcatcaaagagtttctggactatctggaaatcgacttcgaa
atcgtaggagattcctgtatggtctatggggatacccttgatataatgcacggggacctg
gagctttcttcggcagtcgtcgggccagttcctcttgatagggaatggggcattgacaaa
ccatggataggtgcaggttttgggcttgaacgcttgctcaaggttatgcatggctttaaa
aacattaagagagcatcaaggtccgaatcttactataatgggatttcaaccaatctatga

to change the codon for L266 to a codon for M.

In other words the naturally occurring codon for the amino acid corresponding to L266 (which in this sequence is nucleotides 796,797,798 which in this example are CTT) should be changed to the codon for methionine, which is ATG.

This produces a sequence

atggataaaaaaccattagatgttttaatatctgcgaccgggctctggatgtccaggact
ggcacgctccacaaaatcaaacactatgaggtctcaagaagtaaaatatacattgaaatg
gcgtgtggagaccatcttgttgtgaataattctaggagttgtagaacagccagagcattc
agacatcataagtacagaaaaacctgcaaacgatgtagggtttcggacgaggatatcaat
aatttcctcacaagatcaactgaaggcaaaaccagtgtgaaagttaaggtagtttctgct
ccaaaggtcaaaaaagctatgccgaaatcagtttcgagggctccaaagcctctggaaaat
cctgtgtctgcaaaggcatcaacggacacatccagatctgtaccttcgcctgcaaaatca
actccaaattcgcctgttcccacatcggctcctgctccttcacttacaagaagccagctc
gatagggttgaggctctcttaagtccagaggataaaatttctctgaatattgcaaagcct
ttcagggaacttgagtccgaacttgtgacaagaagaaaaaacgattttcagcggctctat
accaatgatagagaagactaccttggtaaactcgaacgggacattacgaaatttttcgta
gaccgggattttctggagataaagtctcctatccttattccggcagaatacgtggagaga
atgggtattaacaatgatactgaactttcaaaacagatcttcagggtggataaaaatctc
tgcttaaggccaatgatggccccgactctttacaactatctgcgaaaactcgataggatt
ttaccagatcctataaagattttcgaagtcgggccctgttaccggaaagagtctgacggc
aaagagcacctggaagaatttaccatggtgaacttctgtcagatgggttcgggatgtact
cgggaaaatcttgaatccctcatcaaagagtttctggactatctggaaatcgacttcgaa
atcgtaggagattcctgtatggtctatggggatacccttgatataatgcacggggacctg
gagctttcttcggcagtcgtcgggccagttcctcttgatagggaatggggcattgacaaa
ccatggataggtgcaggttttgggcttgaacgcttgctcaaggttatgcatggctttaaa
aacattaagagagcatcaaggtccgaatcttactataatgggatttcaaccaatctatga

This can be accomplished by any suitable means known in the art such as site directed mutagenesis, PCR, synthesis of oligonucleotides (with ligation and sequencing as necessary) or other suitable method.

This applies equally to each of the other mutations discussed herein.

### Polypeptides of the Invention

Suitably the polypeptide comprising N^{ε}-acetyl lysine is a nucleosome or a nucleosomal polypeptide.

Suitably the polypeptide comprising N^{ε}-acetyl lysine is a chromatin or a chromatin associated polypeptide.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### Optimisation

Unnatural amino acid incorporation in in vitro translation reactions can be increased by using S30 extracts containing a thermally inactivated mutant of RF-1. Temperature sensitive mutants of RF-1 allow transient increases in global amber suppression in vivo. Increases in tRNA_{CUA} gene copy number and a transition from minimal to rich media may also provide improvement in the yield of proteins incorporating an unnatural amino acid in E. coli.

### INDUSTRIAL APPLICATION

N^{ε}-acetylation regulates diverse cellular processes. The acetylation of lysine residues on several histones modulates chromatin condensation¹, may be an epigenetic mark as part of the histone code², and orchestrates the recruitment of factors involved in regulating transcription, DNA replication, DNA repair, recombination, and genome stability in ways that are beginning to be deciphered³. Over 60 transcription factors and co-activators are acetylated, including the tumor suppressor p53⁴, and the interactions between components of the transcription, DNA replication, DNA repair, and recombination machinery are regulated by acetylations^{5, 6}. Acetylation is important for regulating cytoskeletal dynamics, organizing the immunological synapse and stimulating kinesin transport^{7, 8}. Acetylation is also an important regulator of glucose, amino acid and energy metabolism, and the activity of several key enzymes including histone acetyl-transferases, histone deacetylases, acetyl CoA synthases, kinases, phosphatases, and the ubiquitin ligase murine double minute are directly regulated by acetylation⁹. Acetylation is a key regulator of chaperone function¹⁰, protein trafficking and folding¹¹, stat3 mediated signal transduction¹² and apoptosis¹³. Overall it is emerging that N^{ε}-acetylation is a modification with a diversity of roles and a functional importance that rivals phosphorylation¹⁴. Thus, there are clear utilities and industrial applications for the methods and materials disclosed herein, both in the production of saleable products and in facilitation of the study of essential biological processes as noted above.

### Further Applications

Inhibition of deacetylase may be by any suitable method known to those skilled in the art. Suitably inhibition is by gene deletion or disruption of endogenous deacetylase(s). Suitably such disrupted/deleted acetylase is CobB. Suitably inhibition is by inhibition of expression such as inhibition of translation of endogenous deacetylase(s). Suitably inhibition is by addition of exogenous inhibitor such as nicotinamide.

In one aspect the invention relates to the addition of N^{ε}-acetyl-lysine to the genetic code of organisms such as Escherichia coli.

The invention finds particular application in synthesis of nucleosomes and/or chromatin bearing N^{ε}-acetyl-lysine at defined sites on particular histones. One example of such an application is for determining the effect of defined modifications on nucleosome and chromatin structure and function^{1,26}.

The MbPylRS/MbtRNA_{CUA} pair may be further evolved for the genetic incorporation of mono-, di- and/or tri-methyl-lysine to explore the roles of these modifications on histone structure and function, and/or their role in an epigenetic code¹⁴. Moreover the methods described here may also be applied to genetically incorporate lysine residues derivatized with diverse functional groups and/or biophysical probes into proteins in E. coli.

Since MbPylRS does not recognize the anticodon of MbtRNA_{CUA}¹⁸ it is further possible to combine evolved MbPylRS/MbtRNA pairs with other evolved orthogonal aminoacyl-tRNA synthetase/tRNA_{CUA} pairs, and/or with orthogonal ribosomes with evolved decoding properties²⁷ to direct the efficient incorporation of multiple distinct useful unnatural amino acids in a single protein.

tRNA Synthetases

The tRNA synthetase of the invention may be varied. Although specific tRNA synthetase sequences may have been used in the examples, the invention is not intended to be confined only to those examples.

In principle any tRNA synthetase which provides the same tRNA charging (aminoacylation) function can be employed in the invention. In this invention the key function is charging of tRNA with N^{ε}-acetyl lysine. In this invention the key function is provided by the exemplary L266M substitution.

For example the tRNA synthetase may be from any suitable species such as from archea, for example from Methanosarcina barkeri MS; Methanosarcina barkeri str. Fusaro; Methanosarcina mazei Go1; Methanosarcina acetivorans C2A; Methanosarcina thermophila; or Methanococcoides burtonii. Alternatively the tRNA synthetase may be from bacteria, for example from Desulfitobacterium hafniense DCB-2; Desulfitobacterium hafniense Y51; Desulfitobacterium hafniense PCP1; Desulfotomaculum acetoxidans DSM 771.

Exemplary sequences from these organisms are the publically available sequences. The following examples are provided as exemplary sequences for pyrrolysine tRNA synthetases:

>M. barkeriMS/1-41 9/
Methanosarcina barkeri MS
VERSION Q6WRH6.1 GI: 74501411
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>M. barkeriF/1-419/
Methanosarcina barkeri str. Fusaro
VERSION YP_304395.1 GI: 73668380
MDKKPLDVLISATGLWMSRTGTLHKIKHYEVSRSKIYIEMACGDHLVVNNSRSCRTARAFRHHKYRKTC
KRCRVSDEDINNFLTRSTEGKTSVKVKVVSAPKVKKAMPKSVSRAPKPLENPVSAKASTDTSRSVPSPAK
STPNSPVPTSAPAPSLTRSQLDRVEALLSPEDKISLNIAKPFRELESELVTRRKNDFQRLYTNDREDYLGKLE
RDITKFFVDRDFLEIKSPILIPAEYVERMGINNDTELSKQIFRVDKNLCLRPMLAPTLYNYLRKLDRILPDPIKI
FEVGPCYRKESDGKEHLEEFTMVNFCQMGSGCTRENLESLIKEFLDYLEIDFEIVGDSCMVYGDTLDI
MHGDLELSSAVVGPVPLDREWGIDKPWIGAGFGLERLLKVMHGFKNIKRASRSESYYNGISTNL
>M. mazei/1-454
Methanosarcina mazei Gol
VERSION NP_633469.1 GI: 21227547
MDKKPLNTLISATGLWMSRTGTIHKIKHHEVSRSKIYIEMACGDHLVVNNSRSSRTARALRHHKYRKTCK
RCRVSDEDLNKFLTKANEDQTSVKVKVVSAPTRTKKAMPKSVARAPKPLENTEAAQAQPSGSKFSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>M. acetivorans/1-443
Methanosarcina acetivorans C2A
VERSION NP_615128.2 GI: 161484944
MDKKPLDTLISATGLWMSRTGMIHKIKHHEVSRSKIYIEMACGERLVVNNSRSSRTARALRHHKYRKTCR
HCRVSDEDINNFLTKTSEEKTTVKVKVVSAPRVRKAMPKSVARAPKPLEATAQVPLSGSKPAPATPVSA
PAQAPAPSTGSASATSASAQRMANSAAAPAAPVPTSAPALTKGQLDRLEGLLSPKDEISLDSEKPFRE
LESELLSRRKKDLKRIYAEERENYLGKLEREITKFFVDRGFLEIKSPILIPAEYVERMGINSDTELSKQVFRIDK
NFCLRPMLAPNLYNYLRKLDRALPDPIKIFEIGPCYRKESDGKEHLEEFTMLNFCQMGSGCTRENLEAII
TEFLNHLGIDFEIIGDSCMVYGNTLDVMHDDLELSSAVVGPVPLDREWGIDKPWIGAGFGLERLLKV
MHGFKNIKRAARSESYYNGISTNL
>M. thermophila/1-478
Methanosarcina thermophila, VERSION DQ017250.1 GI: 67773308
MDKKPLNTLISATGLWMSRTGKLHKIRHHEVSKRKIYIEMECGERLVVNNSRSCRAARALRHHKYRKIC
KHCRVSDEDLNKFLTRTNEDKSNAKVTVVSAPKIRKVMPKSVARTPKPLENTAPVQTLPSESQPAPTTPIS
ASTTAPASTSTTAPAPASTTAPAPASTTAPASASTTISTSAMPASTSAQGTTKFNYISGGFPRPIPVQASAP
ALTKSQIDRLQGLLSPKDEISLDSGTPFRKLESELLSRRRKDLKQIYAEEREHYLGKLEREITKFFVDRGFLEIK
SPILIPMEYIERMGIDNDKELSKQIFRVDNNFCLRPMLAPNLYNYLRKLNRALPDPIKIFEIGPCYRKESDG
KEHLEEFTMLNFCQMGSGCTRENLEAIIKDFLDYLGIDFEIVGDSCMVYGDTLDVMHGDLELSSAVV
GPVPMDRDWGINKPWIGAGFGLERLLKVMHNFKNIKRASRSESYYNGISTNL
>M. burtonii/1-416
Methanococcoides burtonii DSM 6242, VERSION YP_566710.1 GI: 91774018
MEKQLLDVLVELNGVWLSRSGLLHGIRNFEITTKHIHIETDCGARFTVRNSRSSRSARSLRHNKYRKPCKR
CRPADEQIDRFVKKTFKEKRQTVSVFSSPKKHVPKKPKVAVIKSFSISTPSPKEASVSNSIPTPSISVVKDEV
KVPEVKYTPSQIERLKTLMSPDDKIPIQDELPEFKVLEKELIQRRRDDLKKMYEEDREDRLGKLERDITEFFV
DRGFLEIKSPIMIPFEYIERMGIDKDDHLNKQIFRVDESMCLRPMLAPCLYNYLRKLDKVLPDPIRIFEIGP
CYRKESDGSSHLEEFTMVNFCQMGSGCTRENMEALIDEFLEHLGIEYEIEADNCMVYGDTIDIMHGD
LELSSAVVGPIPLDREWGVNKPWMGAGFGLERLLKVRHNYTNIRRASRSELYYNGINTNL
>D. hafniense_DC B-2/1-279
Desulfitobacterium hafniense DCB-2
VERSION YP_002461289.1 GI: 219670854
MSSFWTKVQYQRLKELNASGEQLEMGFSDALSRDRAFQGIEHQLMSQGKRHLEQLRTVKHRPALLEL
EEGLAKALHQQGFVQVVTPTIITKSALAKMTIGEDHPLFSQVFWLDGKKCLRPMLAPNLYTLWRELERL
WDKPIRIFEIGTCYRKESQGAQHLNEFTMLNLTELGTPLEERHQRLEDMARWVLEAAGIREFELVTESSV
VYGDTVDVMKGDLELASGAMGPHFLDEKWEIVDPWVGLGFGLERLLMIREGTQHVQSMARSLSYL
DGVRLNIN
>D.hafniense_Y51/1-312
Desulfitobacterium hafniense Y51
VERSION YP_521192.1 GI:89897705
MDRIDHTDSKFVQAGETPVLPATFMFLTRRDPPLSSFWTKVQYQILKELNASGEQLEMGFSDALSRDR
AFQGIEHOLMSQGKRHLEQLRTVKHRPALLELEEGLAKALHQQGFVQVVIPTIITKSALAKMTIGEDH
PLFSQVFWLDGKKCLRPMLAPNLYTLWRELERLWDKPIRIFEIGTCYRKESQGAQHLNEFTMLNLTELGT
PLEERHQRLEDMARWVLEAAGIREFELVTESSVVYGDTVDVMKGDLELASGAMGPHFLDEKWEIVD
PWVGLGFGLERLLMIREGTQHVQSMARSLSYLDGVRLNIN
>D.hafniensePCP1/1-288
Desulfitobacterium hafniense
VERSION AY692340.1 GI: 53771772
MFLTRRDPPLSSFWTKVQYQRLKELNASGEQLEMGFSDALSRDRAFQGIEHQLMSQGKRHLEQLRTV
KHRPALLELEEKLAKALHQQGFVQVVTPTIITKSALAKMTIGEDHPLFSQVFWLDGKKCLRPMLAPNLY
TLWRELERLWDKPIRIFEIGTCYRKESQGAQHLNEFTMLNLTELGTPLEERHQRLEDMARWVLEAAGIRE
FELVTESSVVYGDTVDVMKGDLELASGAMGPHFLDEKWEIFDPWVGLGFGLERLLMIREGTQHVQS
MARSLSYLDGVRLNIN
>D.acetoxidans/1-277
Desulfotomaculum acetoxidans DSM 771
VERSION YP003189614.1 GI: 258513392
MSFLWTVSQQKRLSELNASEEEKNMSFSSTSDREAAYKRVEMRLINESKQRLNKLRHETRPAICALENRL
AAALRGAGFVQVATPVILSKKLLGKMTITDEHALFSQVFWIEENKCLRPMLAPNLYYILKDLLRLWEKPV
RIFEIGSCFRKESQGSNHLNEFTMLNLVEWGLPEEQRQKRISELAKLVMDETGIDEYHLEHAESVVYGET
VDVMHRDIELGSGALGPHFLDGRWGVVGPWVGIGFGLERLLMVEQGGQNVRSMGKSLTYLDG
VRLNI

When the particular tRNA charging (aminoacylation) function has been provided by mutating the tRNA synthetase, then it may not be appropriate to simply use another wild-type tRNA sequence, for example one selected from the above. In this scenario, it will be important to preserve the same tRNA charging (aminoacylation) function. This is accomplished by transferring the mutation(s) in the exemplary tRNA synthetase into an alternate tRNA synthetase backbone, such as one selected from the above.

In this way it should be possible to transfer selected mutations to corresponding tRNA synthetase sequences such as corresponding pylS sequences from other organisms beyond exemplary M. barkeri and/or M. mazei sequences.

Target tRNA synthetase proteins/backbones, may be selected by alignment to known tRNA synthetases such as exemplary M. barkeri and/or M. mazei sequences.

This subject is now illustrated by reference to the pylS (pyrrolysine tRNA synthetase) sequences but the principles apply equally to the particular tRNA synthetase of interest.

For example, FIG. 10 provides an alignment of all PylS sequences. These can have a low overall % sequence identity. Thus it is important to study the sequence such as by aligning the sequence to known tRNA synthetases (rather than simply to use a low sequence identity score) to ensure that the sequence being used is indeed a tRNA synthetase.

Thus suitably when sequence identity is being considered, suitably it is considered across the tRNA synthetases as in FIG. 10. Suitably the % identity may be as defined from FIG. 10. FIG. 2 shows a diagram of sequence identities between the tRNA synthetases. Suitably the % identity may be as defined from FIG. 11.

It may be useful to focus on the catalytic region. FIG. 12 aligns just the catalytic regions. The aim of this is to provide a tRNA catalytic region from which a high % identity can be defined to capture/identify backbone scaffolds suitable for accepting mutations transplanted in order to produce the same tRNA charging (aminoacylation) function, for example new or unnatural amino acid recognition.

Thus suitably when sequence identity is being considered, suitably it is considered across the catalytic region as in FIG. 12. Suitably the % identity may be as defined from FIG. 12. FIG. 4 shows a diagram of sequence identities between the catalytic regions. Suitably the % identity may be as defined from FIG. 13.

‘Transferring’ or ‘transplanting’ mutations onto an alternate tRNA synthetase backbone can be accomplished by site directed mutagenesis of a nucleotide sequence encoding the tRNA synthetase backbone. This technique is well known in the art. Essentially the backbone pylS sequence is selected (for example using the active site alignment discussed above) and the selected mutations are transferred to (i.e. made in) the corresponding/homologous positions.

When particular amino acid residues are referred to using numeric addresses, unless otherwise apparent, the numbering is taken using MbPylRS (Methanosarcina barkeri pyrrolysyl-tRNA synthetase) amino acid sequence as the reference sequence (i.e. as encoded by the publicly available wild type Methanosarcina barkeri PylS gene Accession number Q46E77):

MDKKPLDVLI SATGLWMSRT GTLHKIKHYE VSRSKIYIEM ACGDHLVVNN SRSCRTARAF
RHHKYRKTCK RCRVSDEDIN NFLTRSTEGK TSVKVKVVSA PKVKKAMPKS VSRAPKPLEN
PVSAKASTDT SRSVPSPAKS TPNSPVPTSA PAPSLTRSQL DRVEALLSPE DKISLNIAKP
FRELESELVT RRKNDFQRLY TNDREDYLGK LERDITKFFV DRDFLEIKSP ILIPAEYVER
MGINNDTELS KQIFRVDKNL CLRPMLAPTL YNYLRKLDRI LPDPIKIFEV GPCYRKESDG
KEHLEEFTMV NFCQMGSGCT RENLESLIKE FLDYLEIDFE IVGDSCMVYG DTLDIMHGDL
ELSSAVVGPV PLDREWGIDK PWIGAGFGLE RLLKVMHGFK NIKRASRSES YYNGISTNL

This is to be used as is well understood in the art to locate the residue of interest. This is not always a strict counting exercise—attention must be paid to the context or alignment. For example, if the protein of interest is of a slightly different length, then location of the correct residue in that sequence corresponding to (for example) L266 may require the sequences to be aligned and the equivalent or corresponding residue picked, rather than simply taking the 266th residue of the sequence of interest. This is well within the ambit of the skilled reader.

Notation for mutations used herein is the standard in the art. For example L266M means that the amino acid corresponding to L at position 266 of the wild type sequence is replaced with M.

The transplantation of mutations between alternate tRNA backbones is now illustrated with reference to exemplary M. barkeri and M. mazei sequences, but the same principles apply equally to transplantation onto or from other backbones.

For example Mb AcKRS is an engineered synthetase for the incorporation of AcK

Parental protein/backbone: M. barkeri PylS

Mutations: L266V, L270I, Y271F, L274A, C317F

Mb PCKRS: engineered synthetase for the incorporation of PCK

Parental protein/backbone: M. barkeri PylS

Mutations: M241F, A267S, Y271C, L274M

Synthetases with the same substrate specificities can be obtained by transplanting these mutations into M. mazei PylS. The sequence homology of the two synthetases can be seen in FIG. 14. Thus the following synthetases may be generated by transplantation of the mutations from the Mb backbone onto the Mm tRNA backbone: Mm AcKRS introducing mutations L301V, L305I, Y306F, L309A, C348F into M. mazei PylS, and

Mm PCKRS introducing mutations M276F, A302S, Y306C, L309M into M. mazei PylS.

Full length sequences of these exemplary transplanted mutation synthetases are given below.

>Mb_PyIS/1-419
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mb_AcKRS/1-419
MDKKPLDVLISATGLWMSRTGTLHKIKHHEVSRSKIYIEMACGDHLVVNNSRSCRTARAFRHHKYRKTC
KRCRVSGEDINNFLTRSTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMVAPTIFNYARKLDRILPG
PIKIFEVGPCYRKESDGKEHLEEFTMVNFFQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mb_PCKRS/1-419
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERFGINNDTELSKQIFRVDKNLCLRPMLSPTLCNYMRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mm_PyIS/1-454
MDKKPLNTLISATGLWMSRTGTIHKIKHHEVSRSKIYIEMACGDHLVVNNSRSSRTARALRHHKYRKTCK
RCRVSDEDLNKFLTKANEDQTSVKVKVVSAPTRTKKAMPKSVARAPKPLENTEAAQAQPSGSKFSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>Mm_AcKRS/1-454
MDKKPLNTLISATGLWMSRTGTIHKIKHHEVSRSKIYIEMACGDHLVVNNSRSSRTARALRHHKYRKTCK
RCRVSDEDLNKFLTKANEDQTSVKVKVVSAPTRTKKAMPKSVARAPKPLENTEAAQAQPSGSKFSPAI
PVSTQESVSVPASVSTSISSISTGATASALVKGNINPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMVAPNIFNYARKLDRALPDPIKIFEIGPCYRKESDGKEHLEEFTMLNFFQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>Mm_PCKRS/1-454
MDKKPLNTLISATGLWMSRTGTIHKIKHHEVSRSKIYIEMACGDHLVVNNSRSSRTARALRHHKYRKTCK
RCRVSDEDLNKFLTKANEDQTSVKVKVVSAPTRTKKAMPKSVARAPKPLENTEAAQAQPSGSKFSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERFGIDNDTELSK
QIFRVDKNFCLRPMLSPNLCNYMRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL

The same principle applies equally to other mutations and/or to other backbones.

Transplanted polypeptides produced in this manner should advantageously be tested to ensure that the desired function/substrate specificities have been preserved.

### BRIEF DESCRIPTION OF THE DRAWINGS

The FIGS. 1 to 9 are described in the examples section below under the heading “Figure Legends”.

FIG. 7 is referred to as ‘Supplementary FIG. 1’ Supplementary FIG. 1: A. The two-plasmid system used for histone expression. B. Amino acid sequence of the histones produced from the pCDF PylT plasmids after TEV cleavage.

FIG. 8 is referred to as ‘Supplementary FIG. 2’ Supplementary FIG. 2A. Molecular mass of H3 K14Ac confirmed by electrospray ionization mass spectrometry. His6-tagged Histone H3 K14ac was expressed in E. coli BL21 DE3, purified by Ni2+ chromatography and cleaved with TEV protease. The observed mass of the protein (15299.0 Da) corresponds well to the theoretical mass of a singly acetylated histone H3 (15298.9 Da). Additional peaks of higher mass result from non-covalent phosphate adducts. Supplementary FIG. 2B. Molecular mass of H3 K23Ac confirmed by electrospray ionization mass spectrometry. His6-tagged Histone H3 K23Ac was expressed in E. coli BL21 DE3, purified by Ni2+ chromatography and cleaved with TEV protease. The observed mass of the protein (15299.5 Da) corresponds well to the theoretical mass of a singly acetylated histone H3 (15298.9 Da). Additional peaks of higher mass result from non-covalent phosphate adducts. Supplementary FIG. 2C. Molecular mass of H3 K27Ac confirmed by electrospray ionization mass spectrometry. His6-tagged histone H3 K27ac was expressed in E. coli BL21 DE3, purified by Ni2+ chromatography and cleaved with TEV protease. The observed mass of the protein (15297.4 Da) corresponds well to the theoretical mass of a singly acetylated histone H3 (15298.9 Da). Additional peaks of higher mass result from non-covalent phosphate adducts. Supplementary FIG. 2D: Purification and TEV protease cleavage of histones H2A and H2B. Proteins were expressed in E. coli Rosetta DE3 and purified by Ni2+ chromatography. The proteins were subsequently cleaved with TEV protease (0.08 mg/ml) and analysed on 4-12% SDS-PAGE gels. Samples of each protein are shown before and after TEV cleavage. Supplementary FIG. 2E. Molecular mass of H2A K9Ac confirmed by electrospray ionization mass spectrometry. His6-tagged histone H2A K9Ac was expressed in E. coli Rosetta DE3, purified by Ni2+ chromatography and cleaved with TEV protease. The observed mass of the protein (16327.0 Da) corresponds well to the theoretical mass of a singly acetylated histone H2A lacking the N-terminal methionine (16325 Da). Supplementary FIG. 2F. Molecular mass of H2B K5Ac confirmed by electrospray ionization mass spectrometry. His6-tagged histone H2B K5Ac was expressed in E. coli Rosetta DE3, purified by Ni2+ chromatography and cleaved with TEV protease. The observed mass of the protein (15870.0 Da) corresponds well to the theoretical mass of a singly acetylated histone H2B lacking the N-terminal methionine (15869 Da). Additional peaks of higher mass mainly result from non-covalent phosphate adducts. Supplementary FIG. 2G. Molecular mass of H2B K20Ac confirmed by electrospray ionization mass spectrometry. His6-tagged histone H2B K20Ac was expressed in E. coli Rosetta DE3, purified by Ni2+ chromatography and cleaved with TEV protease. The observed mass of the protein (15871.0 Da) corresponds well to the theoretical mass of a singly acetylated histone H2B lacking the N-terminal methionine (15869 Da). Additional peaks of higher mass mainly result from non-covalent phosphate adducts. Supplementary FIG. 2H: Top down sequencing of histone H2A K9Ac confirms site-specific incorporation of acetyl-lysine. The purified protein was subjected to MALDI top down sequencing as described in the Materials and Methods. The protein sequence inferred from the mass differences of individual ions is indicated above the spectrum and confirms the site-specific incorporation of acetyl-lysine. Supplementary FIG. 2I: Top down sequencing of Histone H2B K5Ac confirms site-specific incorporation of acetyl-lysine. The purified protein was subjected to MALDI top down sequencing as described in the Materials and Methods. The protein sequence inferred from the mass differences of individual ions is indicated above the spectrum and confirms the site-specific incorporation of acetyl-lysine. Supplementary FIG. 2J: Top down sequencing of Histone H2B K20Ac confirms site-specific incorporation of acetyl-lysine. The purified protein was subjected to MALDI top down sequencing as described in the Materials and Methods. The protein sequence inferred from the mass differences of individual ions is indicated above the spectrum and confirms the site-specific incorporation of acetyl-lysine.

FIG. 9 is referred to as ‘Supplementary FIG. 3’ Supplementary FIG. 3. Molecular mass of H2A K119C and its labelling with maleimide-Cy5 confirmed by electrospray ionization mass spectrometry. Histone H2A K119C was expressed in E. coli Rosetta DE3 pLysS, purified as described (Luger et al. 1999) and modified with maleimide-Cy5 (GE Healthcare). The unmodified (red) and the Cy5 labelled (black) proteins were analysed by ESI mass spectrometry. The main peak for the unmodified protein (13925.6 Da) corresponds well to the theoretical mass of histone H2A lacking the N-terminal methionine (13925.1 Da). The labelled histone gave rise to a peak of 14705.2 Da, which corresponds well to the calculated mass of 14703.1 Da. The spectra demonstrate a virtually complete labelling of the histone.

FIG. 10 shows alignment of PylS sequences.

FIG. 11 shows sequence identity of PylS sequences.

FIG. 12 shows alignment of the catalytic domain of PylS sequences (from 350 to 480; numbering from alignment of FIG. 10).

FIG. 13 shows sequence identity of the catalytic domains of PylS sequences.

FIG. 14 shows alignment of synthetases with transplanted mutations based on M. barkeri PylS or M. mazei PylS. The red asterisks indicate the mutated positions.

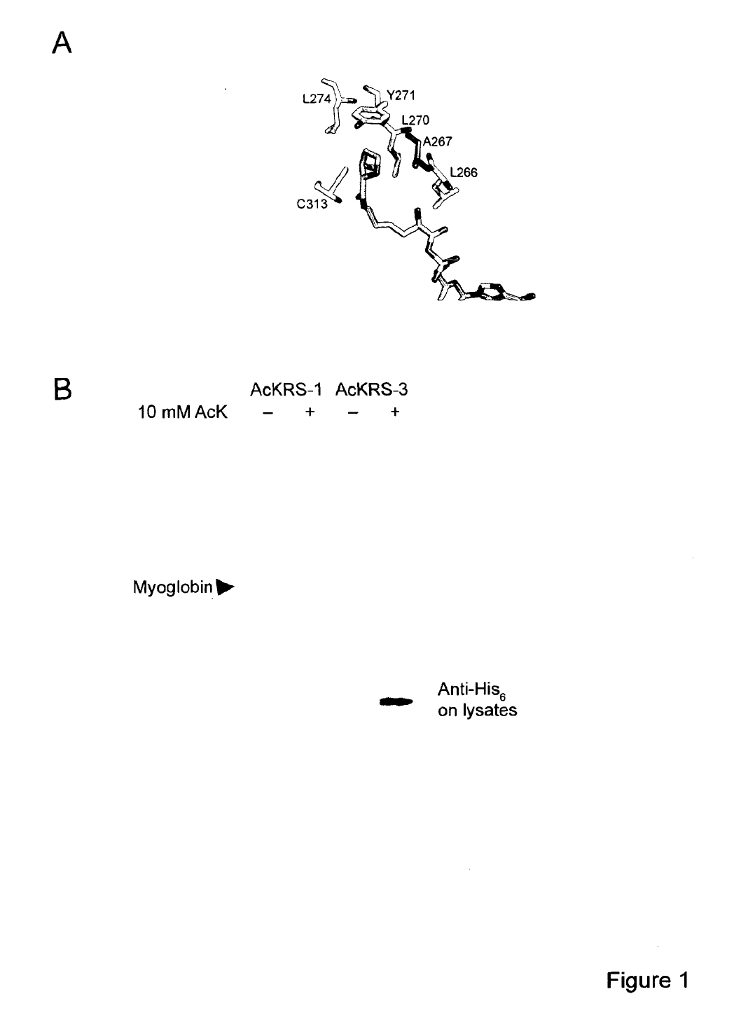

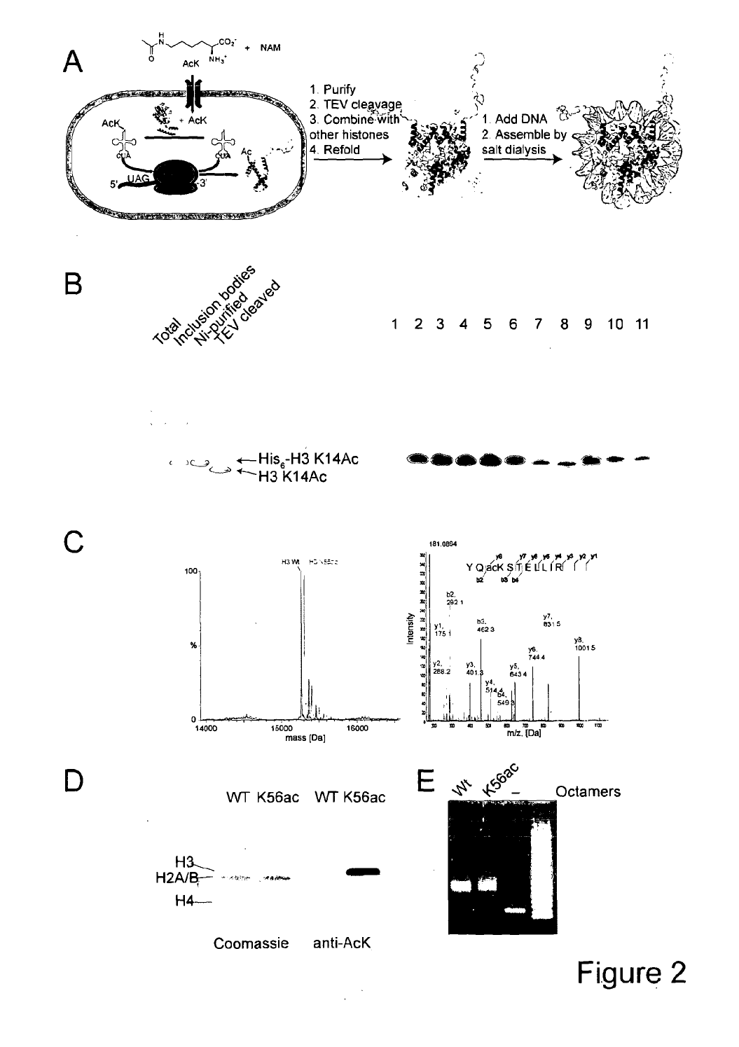

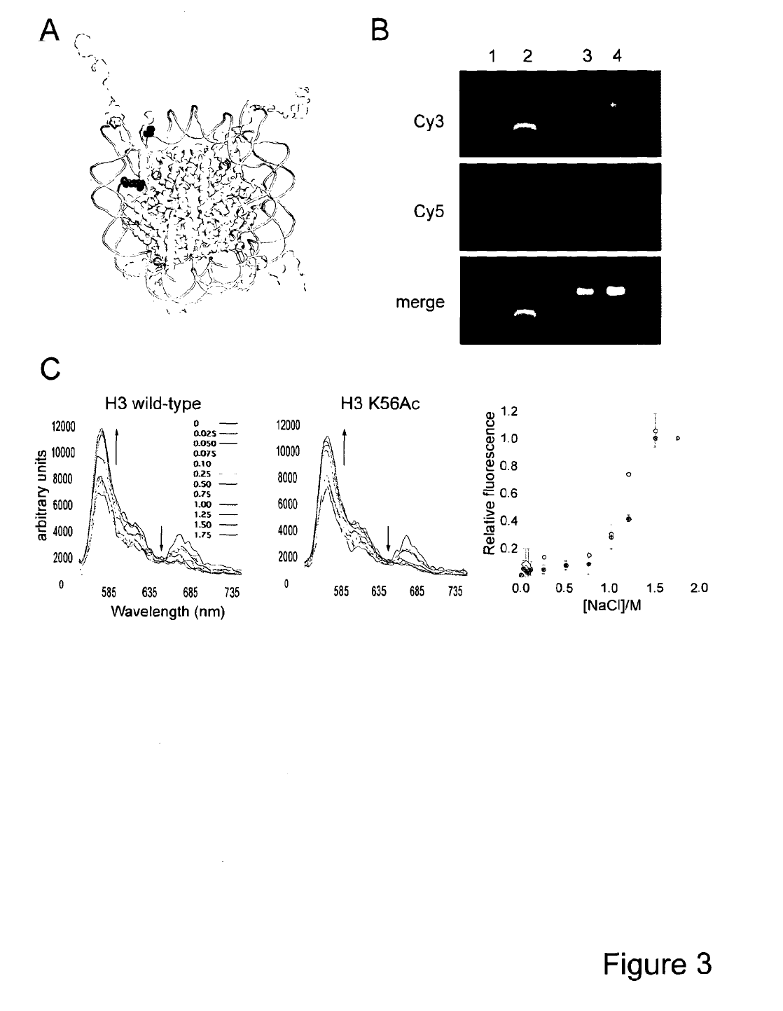

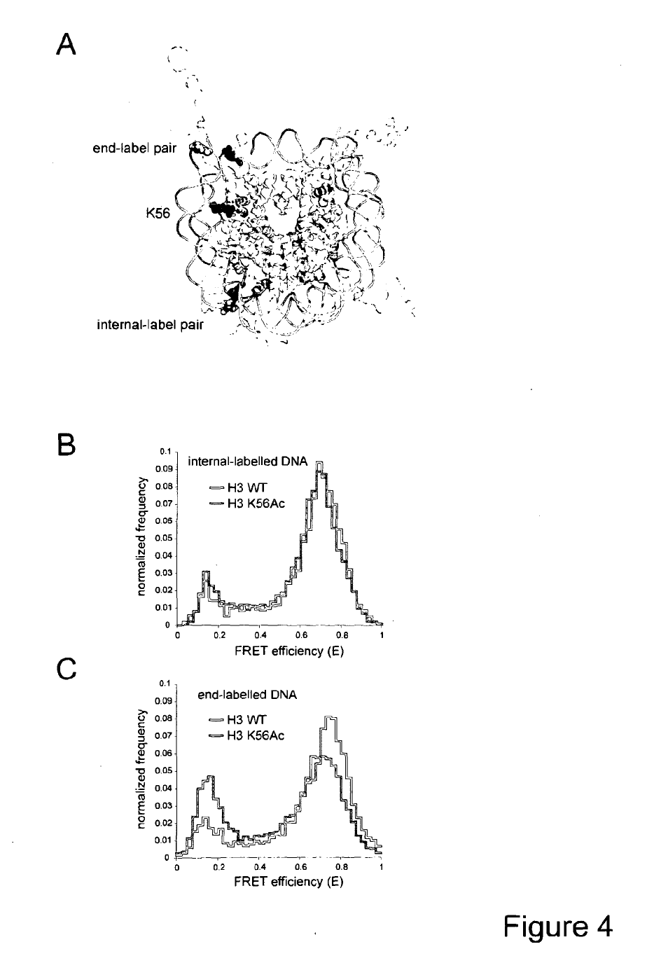

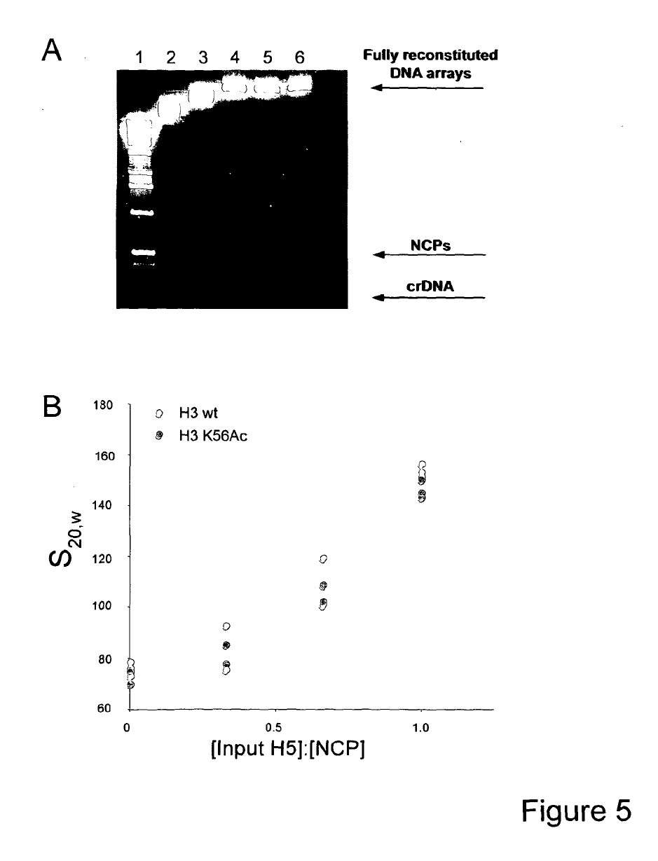

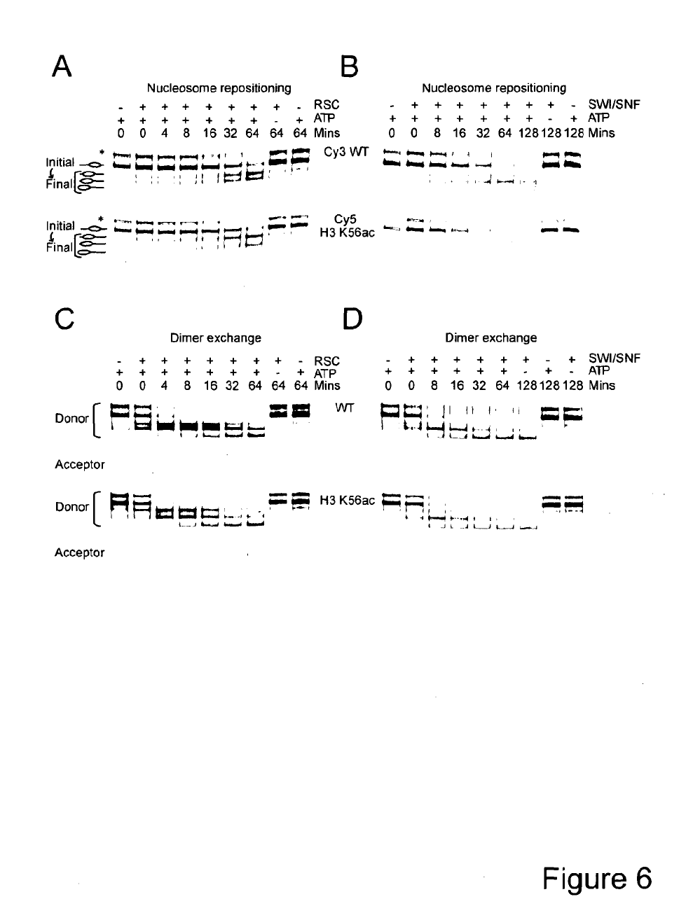

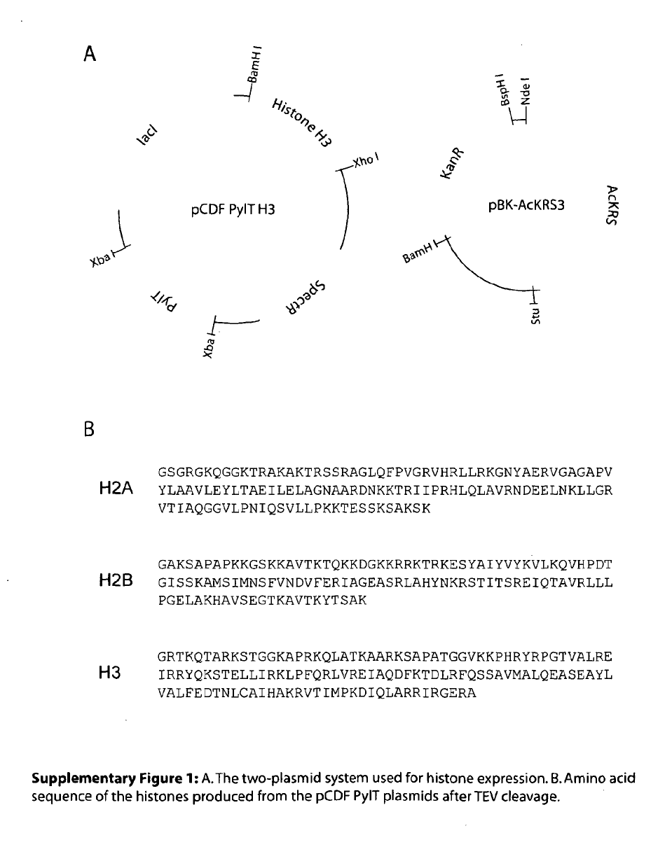

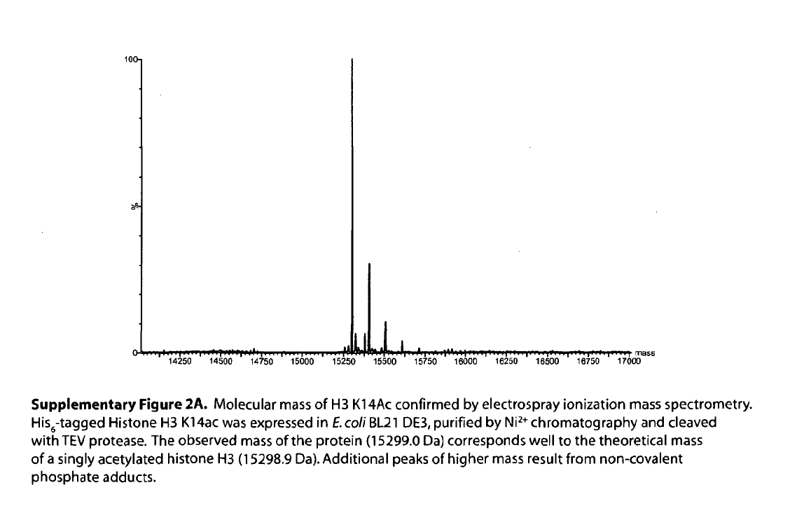

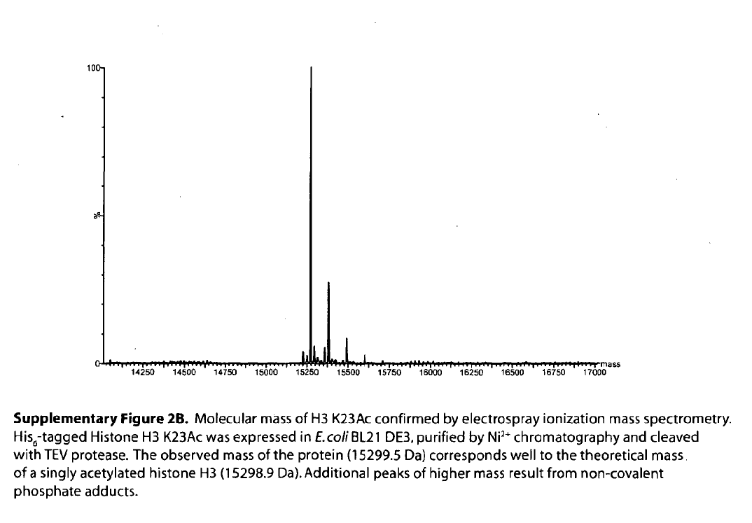

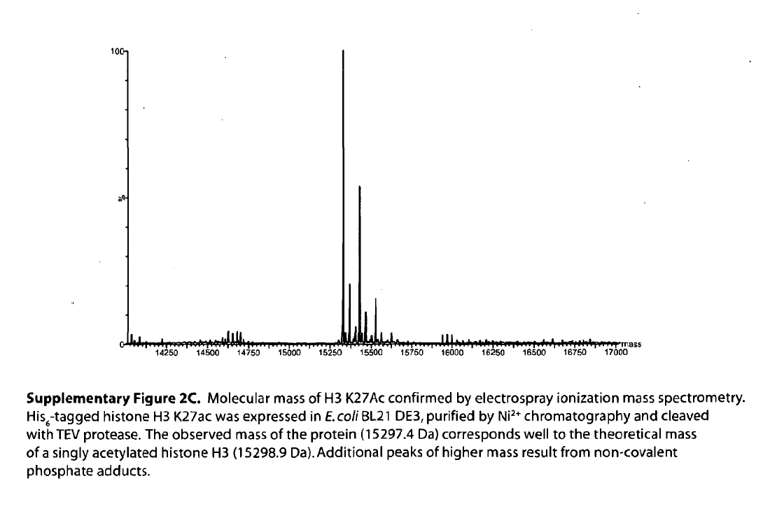

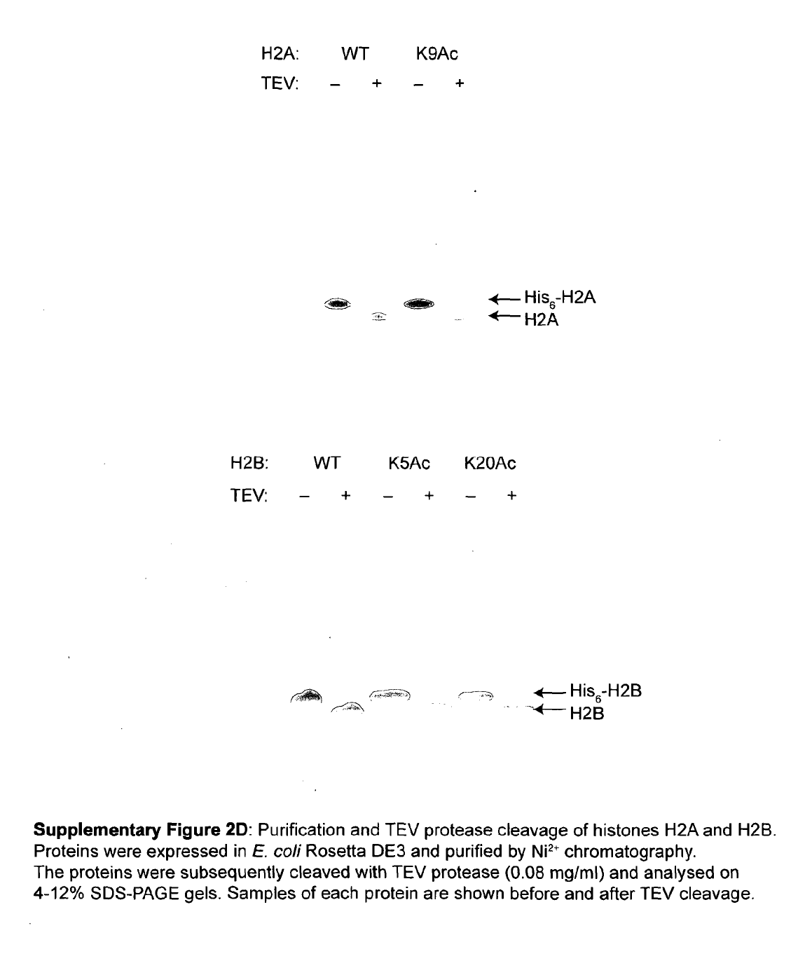

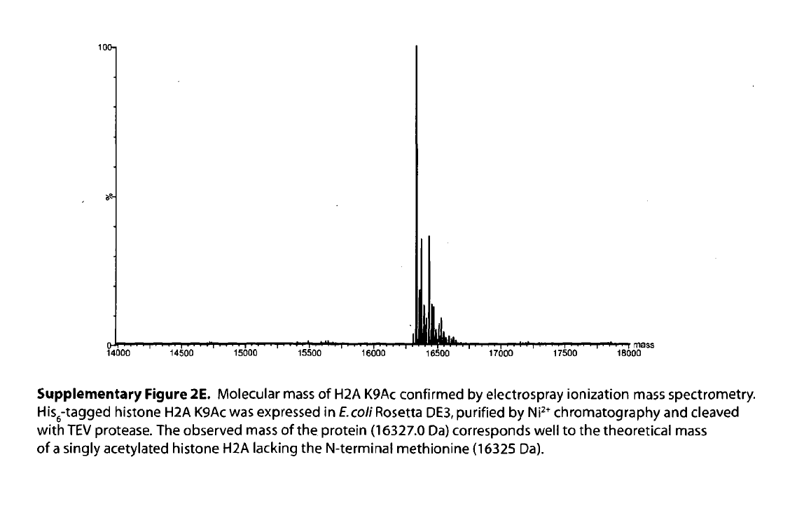

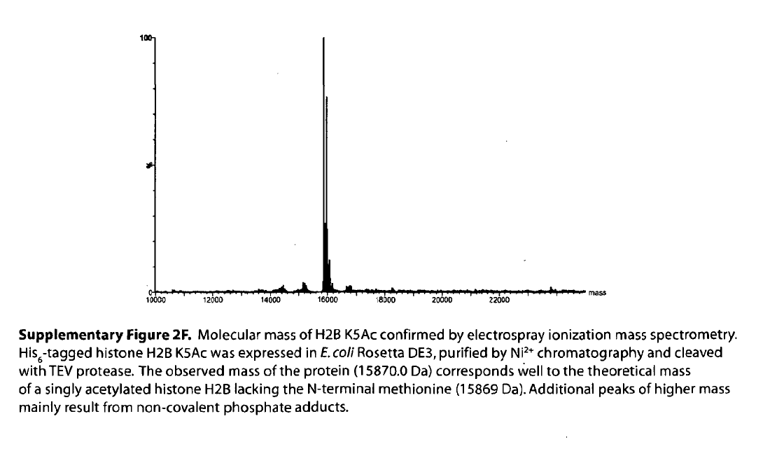

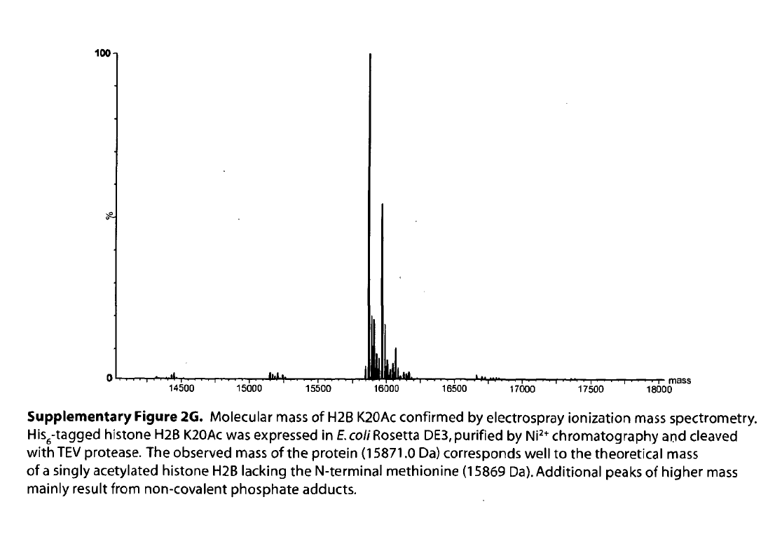

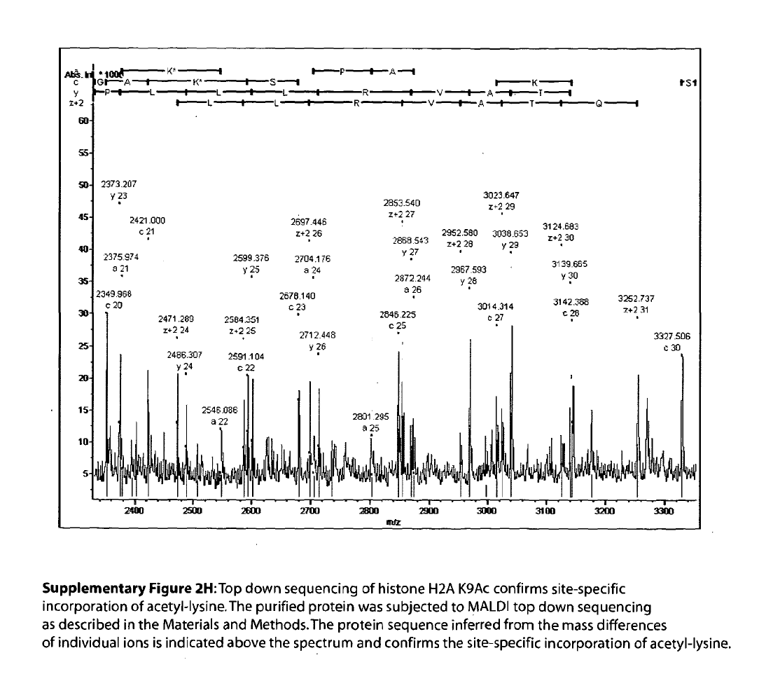

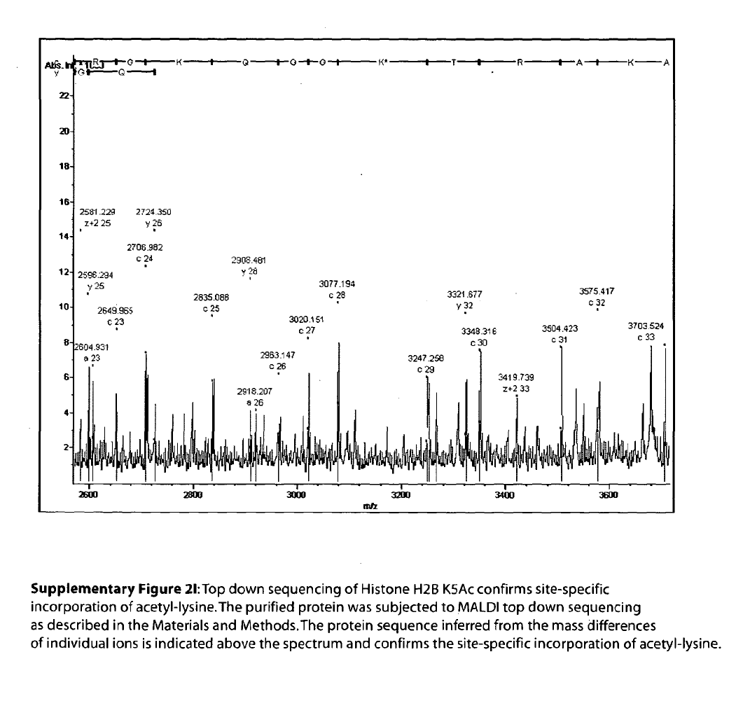

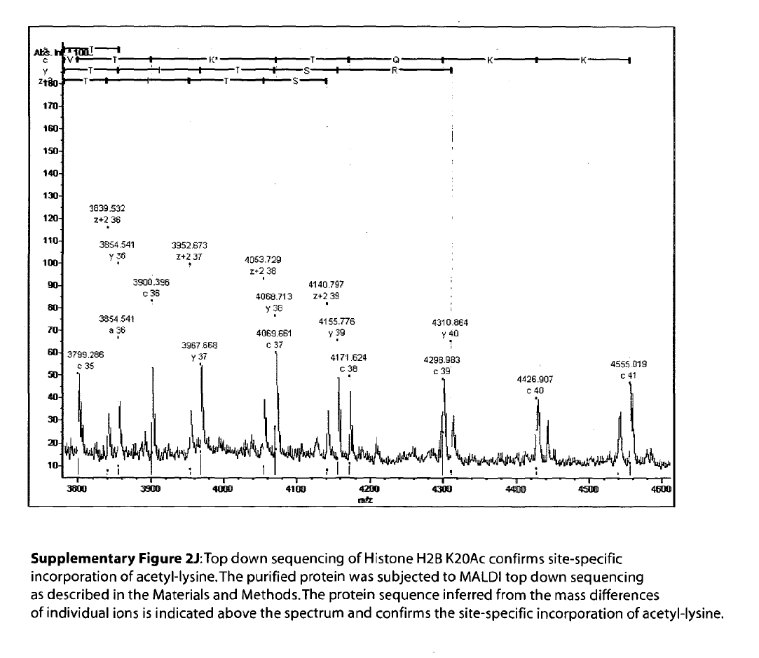

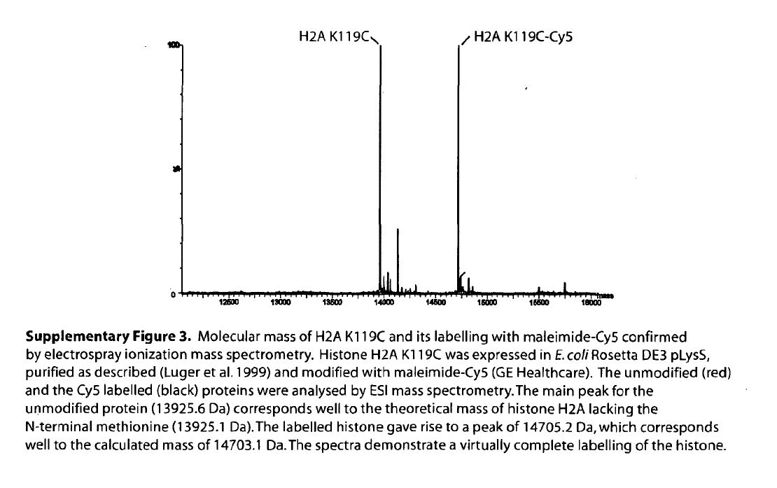

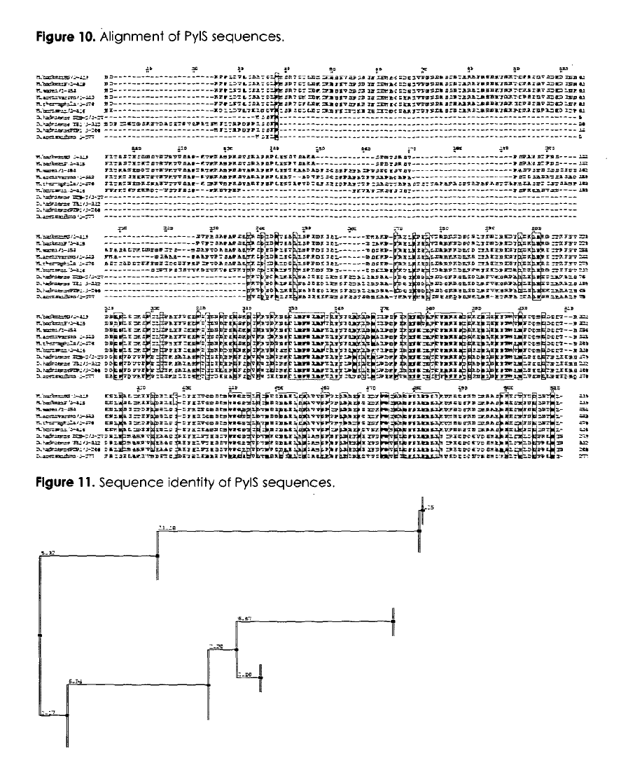

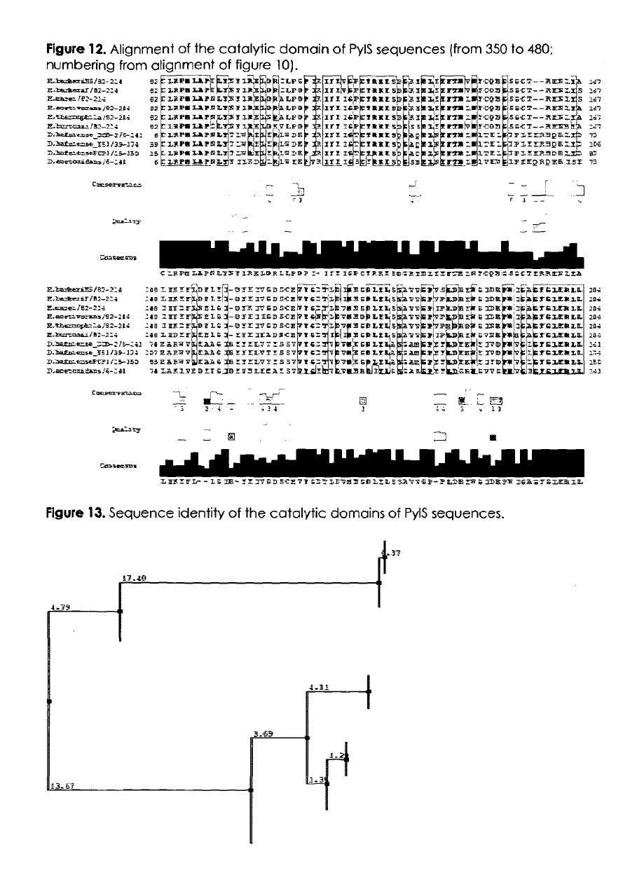

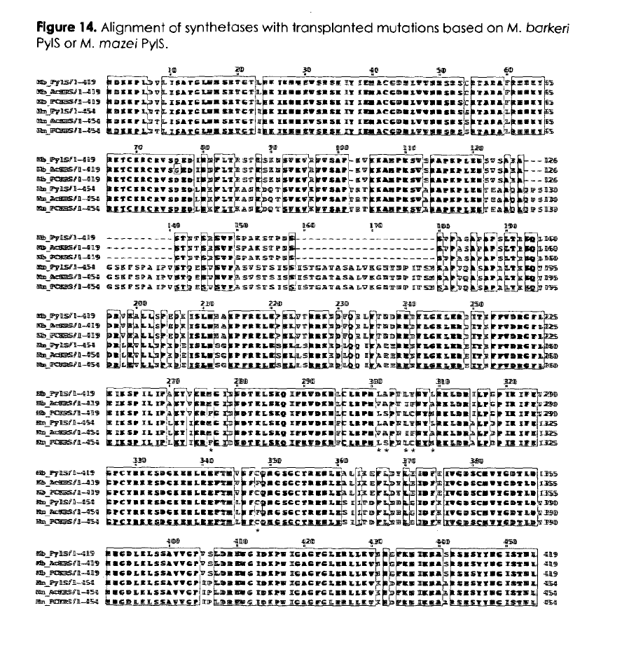

The invention is now described by way of example. These examples are intended to be illustrative, and are not intended to limit the appended claims.

### EXAMPLES

### Summary:

Lysine acetylation of histones defines the epigenetic status of human embryonic stem cells, and orchestrates DNA replication, chromosome condensation, transcription, telomeric silencing, and DNA repair. A detailed mechanistic analysis of these phenomena is impeded by the limited availability of homogeneously acetylated histones. Here we report a general method for the production of homogenously and site-specifically acetylated recombinant histones by genetically encoding acetyl-lysine. We use these histones to reconstitute histone octamers, nucleosomes and nucleosomal arrays bearing defined acetylated lysine residues. With these designer nucleosomes we demonstrate that, in contrast to the prevailing dogma, acetylation of H3K56 does not directly affect the compaction of chromatin, nucleosome stability or remodelling by RSC or SWI/SNF. We observe an increase in DNA breathing in single-molecule FRET experiments, supporting the proposal that deacetylation of H3K56Ac mediates silencing by restricting partial unwrapping of the DNA at the entry-exit point of the nucleosome.

### Introduction

The post-translational acetylation of chromatin on the r-amine of lysine residues in histone proteins defines the epigenetic status of human embryonic stem cells, and is a crucial regulator of DNA replication, chromosome condensation, transcription, and DNA repair in model organisms (Grunstein, 1997; Jenuwein and Allis, 2001; Kouzarides, 2007; Peterson and Laniel, 2004; Shahbazian and Grunstein, 2007; Sterner and Berger, 2000). Acetylation may alter nucleosome or chromatin structure and function directly, or act to recruit other factors to the genome (Jenuwein and Allis, 2001; Kouzarides, 2007) via interaction with bromodomain containing proteins (Yang, 2004) and other potential acetyl-lysine binding modules (Li et al., 2008).

Current methods to introduce acetylation into recombinant histones include enzymatic post-translational modification and native chemical ligation (McGinty et al., 2008; Shogren-Knaak et al., 2006). Enzymatic post-translational acetylation of histones has proved challenging. The acetyl-transferase enzymes are large complexes that are difficult to purify. Moreover enzymatic acetylation does not yield homogeneous samples, as acetylation at the desired site is not quantitative and rarely exceeds 30% (Robinson et al., 2008), and in vitro acetylation at other sites leads to heterogeneous samples. The role of H4K16 in antagonizing chromatin compaction was demonstrated using modified histones produced by native chemical ligation underscoring the utility of methods for synthesizing homogeneously and site-specifically acetylated histones (Shogren-Knaak et al., 2006). However this method remains challenging and requires the synthesis of large quantities of peptide thioester, has not been demonstrated for modifications in the core of histones and yields small quantities of acetylated protein. We recently reported proof-of-principle experiments in which we demonstrated the production of a homogeneously acetylated protein using an aminoacyl-tRNA synthetase and tRNA_{CUA} pair that we created by directed evolution. This pair directs the incorporation of acetyl-lysine into response to an amber codon in the corresponding gene encoded on a plasmid in E. coli (Neumann et al., 2008). While this system is in principle applicable to the production of homogeneously and site-specifically acetylated histones its utility has previously only been demonstrated in a single case with MnSOD, a small non-histone enzyme. Here we develop an improved version of this system and demonstrate that the improved system allows the synthesis of milligram quantities of homogeneously and quantitatively acetylated histones, including histones that are acetylated in the globular core of the protein that cannot be made by any other method.

It is emerging that modifications in the globular core of histones play crucial roles in regulating the structure and function of chromatin and controlling biological function (Cosgrove et al., 2004). H3 K56 acetylation is a particularly important modification in the globular core of H3 (Masumoto et al., 2005; Ozdemir et al., 2005; Xu et al., 2005) that is conserved from yeast to humans (Garcia et al., 2007). Numerous reports have demonstrated its role in DNA repair and replication, regulation of transcription and chromatin assembly (Celic et al., 2006; Celic et al., 2008; Chen et al., 2008; Driscoll et al., 2007; Han et al., 2007; Hyland et al., 2005; Li et al., 2008; Ozdemir et al., 2005; Rufiange et al., 2007; Williams et al., 2008; Xie et al., 2009; Xu et al., 2005; Xu et al., 2007; Yang et al., 2008). The location of K56 on the first helix of H3 close to the DNA at the entry-exit point on the nucleosome (Luger et al., 1997) has led to the postulation that its acetylation acts by modulating nucleosome structure. Though H3 K56 acetylation is clearly important in defining epigenetic status, transcription, replication and repair it has not been possible to experimentally and quantitatively test the mechanistic proposals for how K56 acetylation might affect these complex cellular phenomena (Celic et al., 2006; Chen et al., 2008; Cosgrove et al., 2004; Driscoll et al., 2007; Han et al., 2007; Hyland et al., 2005; Li et al., 2008; Masumoto et al., 2005; Rufiange et al., 2007; Xu et al., 2005; Xu et al., 2007).

Here we develop a method to produce site-specifically and homogeneously acetylated histones. Using this method we produce histones modified in the core for the first time. We apply this method to produce recombinant histone H3 that is site-specifically and homogeneously acetylated on K56. We create nucleosomes and chromatin arrays that bear the modification and investigate the effect of H3 K56 acetylation on nucleosome and chromatin structure and function. We produce milligram quantities of histone H3 that is quantitatively and homogeneously acetylated on K56, demonstrating a simple, scaleable and inexpensive route to large quantities of pure H3 K56Ac. We assemble octamers, nucleosomes, and chromatin fibres bearing this acetylation. Moreover, we examine the effect of H3 K56 acetylation on nucleosome stability, transient unwrapping of DNA from single nucleosomes, chromatin compaction and nucleosome remodelling by SWI/SNF and RSC to test the mechanistic hypotheses on the role of this modification based on cellular experiments.

### Results

We recently described an acetyl-lysyl-tRNA synthetase (AcKRS)/tRNA_{CUA} pair that is derived from the M. barkeri (Mb) Pyrrolysyl tRNA synthetase/tRNA_{CUA} pair (Neumann et al., 2008). The AckRS/tRNA_{CUA} pair directs the incorporation of acetyl-lysine in response to the amber codon with high translational efficiency and fidelity to produce homogenously acetylated protein. Our initial efforts to produce acetylated histones with this original system yielded very little material. To improve the efficiency of this system further we made a library, which randomizes residues L266, A267, L270, Y271, L274 in acetyl-lysyl-tRNA synthetase (FIG. 1) and selected for improved efficiency of acetyl-lysine incorporation as previously described (Neumann et al., 2008). These selections yielded an improved synthetase, which contains a single L266M mutation with respect to AcKRS-1. We named the new synthetase AcKRS-3. Expression of myoglobin-his₆ incorporating acetyl-lysine at position 4 from a myoglobin gene bearing an amber codon at position 4 demonstrates the improvement in protein expression using the AckRS-3/tRNA_{CUA} system.

In order to increase the yield in acetylated histone H3 we further created a vector which contains the MbtRNA_{CUA} gene and an N-terminally hexahistidine tagged histone H3 downstream of a T7 promoter (Supplementary FIG. 1). We introduced an amber codon at position 56 and transformed this vector into BL21 E. coli bearing AcKRS-3. Cells were supplemented with 10 mM acetyl-lysine and 20 mM nicotinamide at OD 0.7, and protein expression induced by addition of IPTG 30 minutes later. Like unmodified histone H3, the H3 K56Ac is over-expressed and found in inclusion bodies (FIG. 2B). Histone H3 K56Ac was purified by denaturing Ni-NTA chromatography with a yield of 2 mg per litre of culture. Subsequent cleavage with TEV protease cleanly removed the N-terminal His-6 tag. Electrospray ionization mass spectrometry (FIG. 2C) demonstrates the homogenous incorporation of a single acetyl-lysine residue and MS/MS confirms that the amino acid is incorporated at the genetically encoded site. By simply moving the position of the amber codon in the H3 gene we have made several other important acetylated variants of H3, including H3 K14Ac, K23Ac and K27Ac. By replacing the H3 gene with the H2A or H2B genes bearing amber codons we have also synthesized and characterized H2B K5Ac and K20Ac, and H2A K9Ac, further demonstrating the generality of the method (FIG. 2B and Supplementary FIG. 2).

We assembled H3 K56Ac, with a comparable efficiency to unmodified H3, into octamers with H2A, H2B and H4 using standard methods of refolding (Luger et al., 1999) indicating that acetylation does not affect octamer formation (FIG. 2D). We used these octamers containing H3 K56Ac to assemble nucleosomes with DNA with a comparable efficiency to octamers containing unacetylated H3 (FIG. 2E), indicating that acetylation does not affect the efficiency of nucleosome formation.

The structure of the nucleosome core particle suggests a water-mediated contact between H3 K56 and the phosphate backbone of the DNA at the entry and exit points (Luger et al., 1997). Several groups have proposed that K56 acetylation affects the stability of the nucleosome or DNA breathing on the nucleosome and suggested that this provides a structural, mechanistic and energetic basis for observed cellular phenomena (Cosgrove et al., 2004; Hyland et al., 2005; Masumoto et al., 2005; Xu et al., 2005). However, there is no experimental data on the effect of this modification on nucleosome stability or DNA breathing. To investigate the effect of H3 K56 on nucleosome stability we first compared the equilibrium stability as a function of NaCl concentration for nucleosomes containing H3 K56Ac and unacetylated H3 by fluorescence resonance energy transfer (FRET) using previously established assays and fluorophore positions (Li and Widom, 2004).

We placed a Cy3 FRET donor on the 5′ end of a 147 bp-DNA nucleosome positioning sequence (Lowary and Widom, 1998), and quantitatively labelled a K119C mutant of H2A with a Cy5 maleimide to create a FRET acceptor (Supplementary FIG. 3). We assembled nucleosomes with the Cy3 labelled DNA and octamers that contained the Cy5 labelled H2A and either H3 K56Ac or unacetylated H3 (FIGS. 3A&B). In each nucleosome there are two Cy5 fluorescently labelled H2A molecules, however only one of these H2A molecules is sufficiently close to the Cy3 on the DNA to contribute significantly to FRET (Li and Widom, 2004). At high NaCl concentrations, where the nucleosome is dissociated, excitation of the Cy3 donor leads to strong emission centred on 565 nm, consistent with Cy3 fluorescence and negligible acceptor emission centred on 670 nm, as expected in the absence of FRET (FIG. 3C). At low NaCl concentrations where the nucleosome is intact, excitation of the Cy3 donor leads to a decreased donor emission at 565 nm and an increased Cy5 acceptor emission at 670 nm consistent with a FRET signal. To assess the stability of H3 K56 acetylated nucleosomes we followed the emission of donor and acceptor fluorophores as a function of [NaCl] for nucleosomes bearing H3 K56 acetylation and nucleosomes bearing unmodified H3 (FIG. 3D). We find that acetylated and non-acetylated nucleosomes show comparable stability to NaCl through a range of concentrations that cover partial unwrapping of the DNA, dissociation of H2A/H2B dimers and dissociation of H3/H4 dimers. These data indicate that acetylation of H3 K56 does not have a substantial effect on nucleosome stability, but the error in the assay does not allow us to distinguish small effects in partial unwrapping of the DNA that result from DNA breathing.

To investigate the partial unwrapping of the DNA resulting from the spontaneous transient breathing of DNA from the histone core (Li and Widom) in more detail and with higher resolution we used a recently developed combination of single pair FRET (spFRET), Alternating Excitation (ALEX) selection and native PAGE (Koopmans et al. submitted for publication; manuscript provided for review). Nucleosomes reconstituted on a nucleosome-positioning element DNA, containing a Cy3B-ATTO647N FRET pair, were separated from free DNA using native PAGE. The nucleosome-containing band was excised from the gel and imaged in a confocal microscope using rapidly alternating green and red excitation. Resulting photon bursts were separated into a green and a red channel. The fluorescence of each nucleosome that diffuses through the focus was analysed for both FRET efficiency and fluorescent label stoichiometry. Finally, a distribution of FRET efficiencies was generated from individual nucleosomes that have both the donor and acceptor label. Using these stringent selections we identified complexes that were folded into nucleosomes and contained both fluorescent labels. By examining this population we were able to accurately assess the transient DNA conformations of individual nucleosomes.

To measure the effect of H3 K56 acetylation on DNA breathing we used a label pair position 6 bp from the exit of the nucleosome (end-label, −6 position), located near the positions labeled in our bulk FRET experiments, and a label pair position 29 bp from the exit of the nucleosome (internal label, −29 position) (FIG. 4A).

For the unmodified nucleosomes 11% of the DNA is unwrapped (FRET efficiency <0.3) at the −29 position and 13% of the DNA is unwrapped at the −6 position (FIG. 4B,C). For H3 K56 acetylated nucleosomes the fraction of nucleosomes in which the DNA is unwrapped at the −6 position is doubled to 28% while the fraction of nucleosomes in which the DNA is unwrapped at the −29 position only increases 3%, from 11% to 14%. These data clearly indicates that H3 K56 acetylation is sufficient to cause a local increase in spontaneous DNA breathing at the entry exit point of the DNA on the nucleosome. This effect may increase the accessibility of nucleosomal DNA to other factors.

Compaction is a pre-requisite for heterochromatin formation. Mutation of K56 to a non-charged residue causes defects in silencing at telomeres (Hyland et al., 2005), where K56 acetylation is normally less abundant (Xu et al., 2007). Moreover failure to deacetylate K56 may lead to defective silencing at telomeres (Xu et al., 2007). These experiments suggest that K56 acetylation may mediate, directly or indirectly, the compaction state of chromatin.

To investigate the direct effect of K56 acetylation on chromatin compaction we reconstituted nucleosome arrays, by assembling octamers containing H3 K56Ac or unmodified H3, on 61 repeats of the 197 base pair nucleosome positioning sequence with increasing amounts of H5 linker histone (FIG. 5A) (Huynh et al., 2005). These chromatin arrays were then folded in 1 mM MgCl₂ and 10 mM TEA, pH 7.4, Sedimentation velocity analysis (FIG. 5B) of the H3 K56Ac arrays reveals a compaction profile—upon addition of H5—that is essentially indistinguishable from that of the arrays bearing wild-type H3. Our results suggest that H3 K56 acetylation has little effect on the compaction of the fibre.

Chromatin immunoprecipitation (CHIP) experiments demonstrate a correlation between K56 acetylation and SWI/SNF recruitment to activated promoters (Xu et al., 2005). Since SWI/SNF contains a bromodomain we investigated the effect of K56 acetylation on the direct recruitment of SWI/SNF. We did not detect any difference in binding of K56 acetylated nucleosomes and non-acetylated nucleosomes to SWI/SNF (using electrophoretic mobility shift assays, data not shown), implying that recruitment of SWI/SNF to K56 acetylated nucleosomes is either context dependent or is mediated by another factor. Similarly we did not observe enhanced binding of the acetylated nucleosomes to RSC or Brf1, which also contain bromodomains (data not shown). These experiments demonstrate that K56 acetylation is not sufficient to directly recruit these remodellers.

It is proposed that K56 acetylation modulates chromatin remodelling by SWI/SNF by facilitating access to the DNA at the entry exit gate (Cosgrove et al., 2004). A H3 K56R mutant fails to recruit SWI/SNF as judged by ChIP and fails to activate histone gene transcription. To investigate the effect of H3 K56 acetylation on remodelling by SWI/SNF and RSC we compared the remodelling of nucleosomes containing H3 K56Ac and unmodified nucleosomes (FIGS. 6A & B). SWI/SNF repositioned H3 K56 acetylated nucleosomes 1.4 fold±0.2 faster than unmodified control nucleosomes (FIG. 6A), whereas RSC repositioned the modified nucleosomes 1.2 fold±0.1 faster than controls (FIG. 6B). This is also consistent with the data obtained using H3 K56Q mutated nucleosomes, which were moved ˜1.3-fold faster than wild-type nucleosomes (data not shown). Collectively, these observations show that histone H3 K56 acetylation has a small effect on nucleosome redistribution by RSC and SWI/SNF. However, given that the effects of K56 acetylation on in vivo transcriptional activation are 2-3 fold (Xu et al., 2005) it is possible that these modest effects contribute to the observed activation.

In addition to moving nucleosomes in cis along DNA, the Snf2 sub-family members have also been shown to displace dimers from nucleosomes (Bash et al., 2006; Bruno et al., 2003). We therefore performed ATP-dependent dimer transfer assays (FIGS. 6C & D) using H3 K56 acetylated nucleosomes to see if this modification was able to influence this specific type of remodelling behaviour. Briefly, wild-type or H3 K56 acetylated nucleosomes were assembled with Cy5 labelled H2A onto Cy3 labelled 54A18 DNA fragments. A chromatin acceptor of wild-type H3/H4 tetramer assembled onto 0W0 DNA fragments was added at ˜3 fold molar excess. Following remodelling, the samples were resolved on a native PAGE gel and the Cy5 histone dimer fluorescence monitored. ATP-dependent dimer transfer for both RSC and SWI/SNF was found not to be greatly affected by the H3 K56 acetylated donor.nucleosomes relative to wild-type nucleosome controls (FIGS. 6C and D). Quantitation of the data revealed that both RSC and SWI/SNF were 1.3 fold more efficient at dimer transfer from H3 K56 acetylated nucleosomes in comparison to wild-type nucleosomes indicating that K56 acetylation has little effect on the dimer exchange with these remodellers.

### Discussion

We have developed the first method for the production of large quantities of histones bearing defined and homogeneous acetylations. This method has allowed us to prepare histone H3 with 100% acetylation at K56 in the globular core for the first time. Using nucleosomes assembled with H3 K56Ac we have measured the effect of H3 K56 acetylation on nucleosome stability and DNA breathing at the entry exit points of the nucleosome. Single molecule FRET experiments demonstrate that H3 K56 acetylation is sufficient to cause a twofold increase in DNA in an open conformation on the nucleosome. These results are consistent with the increased MNase sensitivity of chromatin—bearing an H3 K56Q mutation—isolated from yeast resulting from increased nucleosome breathing (Masumoto et al., 2005). Our data also support the proposal that deacetylation of H3 K56Ac by Sir2 might act to close the entry exit gates of DNA around the histone octamer (Xu et al., 2007). The effect of K56 acetylation on the breathing of DNA on nucleosomes may account for the small effect of the modification on SWI/SNF mediated remodelling and dimer exchange. This twofold increase in DNA breathing begins to provide a mechanistic underpinning for changes in histone gene expression and Snf5 binding observed in ChIP experiments, which are of the same magnitude (Xu et al., 2005).

H3 K56 acetylation has been implicated in silencing at telomeres. The acetylation might directly affect compaction or act to recruit factors that affect the remodelling and compaction of chromatin. Our data demonstrate that H3 K56 acetylation is not sufficient to cause the 2-3 fold changes in compaction observed for H4 K16 (Shogren-Knaak et al., 2006). This suggests that the effect of H3 K56 acetylation on silencing is either dependent on the simultaneous presence of other modifications or on the modification dependent recruitment or action of other factors. Our data suggest that while H3 K56 deacetylation by Sir2 has a small effect on closing the entry exit gate around the nucleosome (Xu et al., 2007) the modification is not sufficient to affect the compaction of chromatin directly.

Understanding the effect of lysine acetylation on transcription, DNA replication, DNA repair requires biochemical analysis of the effect of this modification on the structure and function of chromatin (Ruthenburg et al., 2007; Taverna et al., 2007a). Peptide models have allowed the interactions of histone tail modifications to be investigated (Bannister et al., 2001; Dhalluin et al., 1999; Jacobson et al., 2000; Kasten et al., 2004; Lachner et al., 2001; Li et al., 2006; Wysocka et al., 2006), but these experiments cannot address the direct or indirect effects of modifications on nucleosome structure, the higher order structure of chromatin or the effect of modifications on the interaction with other factors and remodellers within the context of intact chromatin. Moreover peptide models cannot be used to probe the role of the emerging modifications in the globular core of histone proteins, the most prominent of which is perhaps H3 K56 acetylation.

Our method will allow us to investigate the roles of histone acetylation within the same nucleosome as well as the roles of multiple acetylations on a single histone (Wang et al., 2007). The method of acetylation is compatible with native chemical ligation (Dawson et al., 1994; McGinty et al., 2008; Shogren-Knaak et al., 2006), as well as methods for installing methylation analogues (Simon et al., 2007), and it will therefore be possible to introduce acetylation in combination with other histone modifications such as ubiquitylation and methylation on individual histones and nucleosomes. Combining the full arsenal of methods for synthetically installing histone post-translational modifications in chromatin will be increasingly important in defining the combinatorial role of modifications, that are being identified simultaneously on the same molecule (Ruthenburg et al., 2007; Taverna et al., 2007b), in encoding molecular and organismal function.

### Experimental

### Library Design and Selection

The kanamycin resistance gene on plasmid pBK-JYRS was replaced by cloning an ampicillin resistance cassette (amplified by PCR from pJC72 with primers 5′-tgg tca tga tac att caa ata tgt atc cgc tc-3′ and 5′-cga gga tcc tct gac get cag tgg aac gaa aac-3′) into the restriction sites BspHI and BamHI. Subsequently, pBK-AcKRS1amp was created by replacing the open reading frame of MjYRS with the NdeI/StuI fragment from plasmid pBK-AcKRS1 containing the ORF of AcKRS1. This plasmid was then used as a template in the generation of a library of PylS mutants. A single round of inverse PCR (Rackham and Chin, 2005) (with primers 5′-gcg cag gtc tca ccg atg DTK NNK ccg acc DTK HWK aac tat NYK cgt aaa ctg gat cgt att ctg ccg ggt c-3′ and 5′-gcg cag agt agg tct cat egg acg cag gca cag gtt ttt atc cac gcg gaa aat ttg-3′) was performed to partially randomize codons for L266 and L270 (to F, L, I, M and V), Y271 (to F, L, I, M, Y, H, Q, N and K) and L274 (to F, L, I, M, V, S, P, T, A). The codon for A267 was mutated to decode all 20 natural amino acids in this library. The PCR product was first digested with DpnI and BsaI and then re-circularized by ligation. Transformation of electro-competent DH10B with the ligation produced 10⁸ transformants, covering the theoretical diversity of the library (2.2×10⁵) by more than 99.99%. Selection of mutants specific for acetyl-lysine was carried out as described. Eventually, the ORF of AcKRS-1 in the original pBKAcKRS-1 plasmid was replaced with AcKRS-3 using the restriction sites NdeI and StuI.

### Expression and Purification of Acetylated Histones H2A, H2B and H3

BL21 DE3 (for H3) or Rosetta DE3 (for H2A and H2B) cells were transformed with plasmid pAcKRS-3 and pCDF PylT-1 carrying the ORF for the histone with amber codons at the desired sites. The cells were grown over night in LB supplemented with 50 μg/ml kanamycin and 50 μg/ml spectinomycin (LB-KS). One litre prewarmed LB-KS was inoculated with 50 ml over night culture and incubated at 37° C. At OD600 of 0.7-0.8 the culture was supplemented with 20 mM nicotinamide (NAM) and 10 mM acetyl-lysine (AcK). Protein expression was induced 30 min later by addition of 0.5 mM IPTG. Incubation was continued at 37° C. and cells were harvested 3-3.5 h after induction, washed with PBS-20 mM NAM and stored at −20° C.

The pellet was resuspended in 30 ml PBS supplemented with 20 mM NAM, 1 mM PMSF, 1×PIC (Roche), 1 mM DTT, 0.2 mg/ml lysozyme and 0.05 mg/ml DNase I and incubated for 20 min with shaking at 37° C. Cells were lysed by sonication (Output level 4 for 2 min on ice). Extracts were clarified by centrifugation (15 min, 18,000 rpm, SS34) and the pellet resuspended in PBS supplemented with 1% Triton X-100, 20 mM NAM and 1 mM DTT. The samples were centrifuged as above and washed again, once with the same buffer then twice with the same buffer without Triton X-100. The pellet was macerated in 1 ml DMSO and incubated for 30 min at room temperature. 25 ml of 6 M guanidinium chloride, 20 mM Tris, 2 mM DTT pH 8.0 were used to extract the histone proteins from the pellet. The samples were incubated for 1 h at 37° C. with shaking, centrifuged as above and loaded onto a preequilibrated Ni²⁺-NTA column (Qiagen). The column was washed with 100 ml 8 M urea, 100 mM NaH₂PO₄, 1 mM DTT pH 6.2. Bound proteins were eluted with 7 M urea, 20 mM sodium acetate, 200 mM NaCl, 1 mM DTT pH 4.5.

### TEV Cleavage

The eluates containing the protein were combined and dialysed at 4° C. against 5 mM β-mercaptoethanol (two times against the 100 fold volume). The solution was made up to 50 mM Tris/HCl pH 7.4 and supplemented 1:50 with 4 mg/ml TEV. The reaction was incubated for 5 h at 30° C. Afterwards, salts were removed by dialysis as above and the protein lyophilized.

### Mass Spectrometry

A 3 μM solution of H3 K56ac in 100 mM (NH₄)HCO₃ was digested with trypsin overnight. An aliquot of this digest was separated by nanoscale liquid chromatography (LC Packings) on a reversed-phase C18 column (150×0.075 mm internal diameter, flow rate 0.25 μl min⁻¹). The eluate was introduced directly into a Q-STAR pulsar i hybrid tandem mass spectrometer (MDS Sciex). The spectra were searched against a NCBI non-redundant database with MASCOT MS/MS Ions search (www.matrixscience.com). The doubly charged ion with m/z 646.88 matched to the acetylated peptide. The acetylation site was confirmed by manual inspection of the fragmentation series.

Protein total mass was determined on an LCT time-of-flight mass spectrometer with electrospray ionization (ESI). (Micromass). Proteins were rebuffered to 20 mM (NH₄)HCO₃ pH 7.5 and diluted 1:100 into 50% methanol, 1% formic acid. Samples were infused into the ESI source at 10 ml min⁻¹, using a Harvard Model 22 infusion pump (Harvard Apparatus) and calibration performed in positive ion mode using horse heart myoglobin. 60-80 scans were acquired and added to yield the mass spectra. Molecular masses were obtained by deconvoluting multiply charged protein mass spectra using MassLynx version 4.1 (Micromass). Theoretical molecular masses of wild-type proteins were calculated using Protparam (http://us.expasy.org/tools/protparam.html), and theoretical masses for unnatural amino acid containing proteins adjusted manually. Where indicated protein total mass and acetylation position sequencing was performed using a top down approach, in these cases in-source decay (ISD) spectra were acquired in reflectron mode on an Ultraflex III TOF/TOF mass spectrometer (Bruker Daltonics, Bremen, Germany) using a 2,5-dihydroxy benzoic acid matrix.

### Histone Octamer Reconstitution

Lyophilized histones were dissolved at an equivalent of 1 mg H2A per ml in unfolding buffer (7 M guanidinium chloride in 20 mM Tris, pH 7.4, 10 mM DTT) and mixed in stoichiometric amounts (Luger et al., 1999). A 2 ml reaction was incubated for 3 h at room temperature with gentle agitation and dialysed against three times 250 ml refolding buffer (2 M NaCl, 10 mM Tris pH 7.4, 1 mM EDTA, 5 mM β-mercaptoethanol) at 4° C. Precipitates were removed by centrifugation (5 min, 25000 g, 2° C.) and filtered using a SpinX column. Octamers were then separated by gel filtration using a Superdex200 column equilibrated with refolding buffer.

Labelling of H2A K119C with Maleimide-Cy5

The K119C mutation was introduced into pET3 H2A by Quikchange and H2A K119C was expressed and purified following published procedures (Luger et al., 1999). The protein was rebuffered to degassed PBS containing 1 mM TCEP using a PD10 column. In a 1 ml reaction 2 mg of the protein were reacted with 400 μg maleimide-Cy5 for 18 h at 4° C. The reaction was then dialysed against two times 500 ml 5 mM β-mercaptoethanol over night at 4° C. and lyophilized. Analysis by ESI-TOF MS showed that the reaction had gone to completion (See supplementary information).

### Measuring Equilibrium Stability of Nucleosomes by FRET

Using the high-affinity nucleosome positioning sequence 601 (Lowary and Widom, 1998) as a template, the 147 bp dominant nucleosome position (Dorigo et al., 2003) on the 282 bp sequence 601 was generated with the following primers: Cy3-LE19: 5′-(Cy3-C)TG GAG AAT CCC GGT GCC G-3′, RE23: 5′-ACA GGA TGT ATA TAT CTG ACA CG-3′ to produce Cy3-labelled 147 bp 601 DNA. The PCR was followed by agarose gel until the oligonucleotide primers were exhausted.

Histone octamers containing unacetylated H3 or H3 K56Ac and Cy5-labelled H2A were reconstituted as described above. Octamers and 147 bp Cy3-DNA were mixed in high-salt buffer (2 M NaCl, 10 mM Tris-HCl (pH 7.4), 1 mM EDTA, 5 mM β-mercaptoethanol) and nucleosome core particles assembled by a continuous dialysis method in which the NaCl concentration was reduced from 2.0 M to 10 mM over a 16 hour period at 4° C. The stoichiometry of histone octamer binding was assessed by gel mobility-shift assays in 0.8% (w/v) agarose gels imaged with a Typhoon Imager. Fluorescence experiments were carried out at room temperature (˜23° C.) on a Tecan safire² spectrophotometer. Nucleosome samples were excited at 515 nm and emission spectra were collected from 535-750 nm. Emission wavelength maxima were observed at 565 nm for Cy3 and 670 nm for Cy5. Samples were incubated for at least 5 minutes at each salt concentration prior to each reading, as it has been previously demonstrated that longer incubation does not lead to any further change in emission intensity (Park et al., 2004), indicating that an equilibrium has been achieved within 5 min. All samples contained a final concentration of ˜8 nM nucleosome core particles. Relative fluorescence intensity was calculated from FRET donor intensity/FRET acceptor intensity and data were normalized using the upper and lower plateau values as baselines.

### Single Molecule FRET

Mononucleosomes were reconstituted on a fluorescently labelled 155 bp-DNA template containing a 601 nucleosome positioning sequence as described (Koopmans et al., 2007). Briefly, the template DNA was prepared by PCR and was labelled with Cy3B (donor) and ATTO647N (acceptor) by incorporation of fluorescently labelled, HPLC purified primers (IBA GmbH, Göttingen, Germany). Nucleosome reconstitutions were analyzed with 5% native poly-acrylamide gel electrophoresis (PAGE). A sample of 0.1-1 pmol was loaded on the gel (29:1 bis:acrylamide, 0.2×TB). The gel was run at 19 V/cm at 4° C. for 80 min and visualized with a gel imager (Typhoon 9400, GE, Waukesha, Wis., USA). The band corresponding to reconstituted nucleosomes was excised and put on a home-built confocal microscope equipped with a 60× water-immersion microscope objective (NA=1.2, Olympus, Zoeterwoude, The Netherlands) and two single photon avalanche photodiodes (SPCM AQR-14, Perkin-Elmer (EG&G), Waltham, Mass., USA). The photodiodes were read out with a TimeHarp 200 photon counting board (Picoquant GmbH, Berlin, Germany). A 515 nm diode pumped solid state laser (Cobolt, Solna, Sweden) and a 636 nm diode laser (Power Technology, Little Rock, Ark., USA) were alternated at 20 kHz for excitation. In a typical experiment, data was collected for 10 min and 1000-5000 bursts of fluorescence were detected.

Photon arrival times in the donor and acceptor channel were sorted according to excitation period, resulting in four photon streams: I₅₁₅^{D}, donor emission during green excitation; I₅₁₅^{A}, acceptor emission during green excitation; I₆₃₆^{D}, donor emission during red excitation; I₆₃₆^{A}, acceptor emission during red excitation. The total fluorescence emission was analyzed with a burst detection scheme as described (Eggeling et al., 2001). A burst was selected if a minimum of 100 photons arrived subsequently, with a maximum interphoton time of 100 μs. For each burst we calculated the apparent FRET efficiency E (also known as proximity ratio):

$$E = \frac{{N}_{515}^{A}}{{N}_{515}^{A} + γ {N}_{515}^{D}} ,$$

and the apparent label stoichiometry S:

$$E = \frac{{N}_{515}^{A} + γ {N}_{515}^{D}}{{N}_{515}^{A} + γ {N}_{515}^{D} + {N}_{636}^{A}} ,$$

where N₅₁₅^{D}, N₅₁₅^{A}, and N₆₃₆^{A} are number of photons in the burst from the different photon streams, and y is a parameter to correct for photophysical properties of the dyes, in our case equal to unity. The excitation powers were chosen such that N₅₁₅^{A}+g N₅₁₅^{D}≈N₆₃₆^{A} resulting in S˜0.5 for doubly labelled nucleosomes. Only nucleosomes with 0.2<S<0.7 were selected for FRET analysis.

### Production of DNA Arrays for Compaction

To produce and purify the DNA arrays, E. coli DH5a containing a pUC18 vector with the DNA array insert was grown overnight in 1 L of LB (37° C., 250 rpm). For blunt-ended release, multimer arrays (2 kbp-15 kbp) were excised by digestion with EcoRV. The vector was digested into smaller products (<1 kbp) using HaeII and DraI. The array DNA was separated from the fragments by selective polyethylene glycol (PEG) precipitation of long DNA fragments using 5-8% PEG 6000 in 0.5 M NaCl. The purified array DNA was phenol/chloroform extracted, ethanol precipitated, and the DNA pellets were re-suspended in 2 M NaCl, 10 mM TEA and 1 mM EDTA.

Competitor DNA (crDNA) was obtained from chicken erythrocyte nuclei. Mono-nucleosomes with approximately 147 bp of mixed sequence DNA were obtained by limited micrococcal nuclease digest of long chicken chromatin. Phenol/chloroform extraction removed bound histones.

### Reconstitution of Nucleosome Arrays

Nucleosome arrays were reconstituted at 25 μg/ml DNA using our in vitro reconstitution method (Huynh et al., 2005). The molar input ratio of histone octamer required to obtain saturation was empirically determined. For compaction studies, the linker histone (H5 or H1) was added to the reconstitution in increasing concentrations. Mixed sequence crDNA (˜147 bp) was added in all reconstitutions at a crDNA:601 DNA array mass ratio of 1:2 to prevent super-saturation of the 601 DNA arrays with excess histone octamer, ensuring that one histone octamer was bound per 601 DNA repeat. Following reconstitution, chromatin arrays were dialysed into folding buffer containing either 1.66 mM MgCl₂ or increasing concentrations of NaCl in 10 mM TEA pH 7.4. The recombinant chromatin arrays—whether acetylated or not—precipitated very easily at higher divalent cation concentration, regardless of the concentration of monovalent ion used. The reconstitution and folding of nucleosome arrays was monitored by electrophoresis in native agarose gels.

### Sedimentation Velocity Analysis Data

Sedimentation velocity analysis data were obtained using a Beckman XL-A analytical ultracentrifuge equipped with scanner optics. Optical density was measured at 260 nm with an initial absorbance between 0.5 and 1.2. Sedimentation analysis was carried out for 2 h at 5° C. at speeds between 15,000 and 22,000 r.p.m. in 12 mm double-sector cells and a Beckman AN60 rotor. Prior to analysis, samples and blanking buffer were placed in cells to settle for approximately one hour as this dramatically improved reproducibility of results. Sedimentation coefficients were determined using the time-derivative method described by Stafford (Stafford, 1992), using John Philo's Dcdt+ data analysis program (version 2.05) (Philo, 2006). Sedimentation coefficients were corrected to S_{20.w}. Partial specific volumes were calculated for all sample assuming values of 0.725 and 0.55 for protein content and DNA content respectively. Partial specific volumes are thus adjusted to account for different nucleosome repeat lengths and linker histone content. Solvent viscosity and solvent density were corrected according to buffer composition.

### Purification of Remodeling Complexes

Yeast strains TAP tagged for RSC (Saha et al., 2002) and SWI/SNF (Chandy et al., 2006) were purified as described previously (Ferreira et al., 2007). The SWI/SNF used for testing H3 K56 acetylated nucleosomes was a kind gift from Salma Mahmood and was purified essentially as described (Ferreira et al., 2007) but with the following changes: 6 L of cells were grown in 1× yeast extract, peptone, adenine, D-glucose. The cells were disrupted using 0.5 mm glass beads in a Bead Beater (Biospec Products Incorporated) using 10 pulses of 30 s ON, 1 min OFF. SWI/SNF wash and storage buffers contained 150 mM NaCl.

### Mononucleosome Repositioning Assays

Nucleosomes were assembled onto DNA fragments described with the nomenclature aBc, with a and c are numbers that describe the length of the upstream and downstream by extensions, respectively. B is the nucleosome positioning sequence source, with A and W representing the mouse mammary tumor virus (MMTV) nucleosome A (Flaus and Richmond, 1998) and 601.3 sequence (Anderson et al., 2002), respectively. Fluorescently labelled oligos were from Eurogentec (Belgium) and unlabelled oligos from the Oligonucleotide Synthesis Laboratory (University of Dundee, UK). The oligo sequences to amplify the 54A18 fragment are 5′-TAT GTA AAT GCT TAT GTA AAC CA-3′ and 5′-TAC ATC TAG AAA AAG GAG C-3′; for the 54A0 fragment 5′-TAT GTA AAT GCT TAT GTA AAC CA-3′ and 5′-ATC AAA ACT GTG CCG CAG-3′; and for the 0W0 fragment 5′-CTG CAG AAG CTT GGT CCC-3′ and 5′-ACA GGA TGT ATA TAT CTG-3′. The PCR was purified by ion exchange chromatography using a 1.8 ml SOURCE 15Q (GE Healthcare) column.

Nucleosomes were assembled on 54A18 DNA fragments for RSC and SWI/SNF repositioning. Each 10 μl reaction contained 1 pmol of wild-type and mutant nucleosomes assembled on Cy3 and Cy5 labelled DNA, respectively, 50 mM NaCl, 50 mM Tris pH 7.5, 3 mM MgCl₂, 1 mM ATP and the quantity of remodeller specified in figures. Samples were incubated in 0.2 ml thin-walled PCR tubes (ABgene, UK) in an Eppendorf mastercycler with heated lid at 30° C. for various time points, before reaction termination by transferal to ice and addition of 500 ng of HindIII-digested bacteriophage lambda competitor DNA (Promega, USA) and 5% (w/v) sucrose. Samples were resolved on a native PAGE gel (5% acrylamide:bis acrylamide (49:1 ratio), 0.25×TBE buffer (0.5 mM EDTA, 22.3 mM Tris-borate, pH 8.3), 0.1% APS and 0.1% TEMED). Gels were cast horizontally between 20 by 20 cm glass plates using 1.5 mm Teflon spacers, before mounting vertically in the gel apparatus (Thermo Fischer Scientific, USA) and pre-running at 300 V for 3 h with continuous pump recirculation of 0.2×TBE buffer between the upper and lower compartments at 4° C. Gels were run at 300 V for 3.5 h and imaged using a Phosphoimager FLA-5100 (Fujifilm, Japan). Gel band intensities were quantitated using AIDA software (Raytest, Germany) and the remodeller repositioning at each time point calculated from the intensity of the sum of all end positions relative to the sum of the major initial and all end positions. The initial rate was calculated as previously described (Ferreira et al., 2007). Each initial rate was repeated at least three times using chromatin prepared in separate assembly reactions.

### Dinner Exchange Assays

Histone H2A T10C was fluorescently labelled by a Cy5 mono maleimide dye (GE Healthcare). Donor nucleosomes were produced by assembly of tetramers and Cy5 labelled dimers onto Cy3 labelled 54A18 DNA fragments. To measure nucleosome assembly efficiency 2 pmol of each assembly reaction was resolved by native PAGE and the assembly quantified by measuring the summed intensity of all nucleosome bands relative to 1 pmol of Cy3 labelled 54A18 DNA. Each 10 μl reaction contained 0.25 pmol of donor nucleosome, 0.75 pmol (3 fold excess) wild-type tetrasome acceptor assembled on 0W0 DNA fragments, 50 mM NaCl, 50 mM Tris pH 7.5, 3 mM MgCl₂, 1 mM ATP and the quantity of remodeller specified in FIG. 6. Reactions were incubated in an Eppendorf mastercycler with heated lid at 30° C. for the specified times. Reactions were terminated by transfer to ice and the addition of 500 ng of HindIII-digested bacteriophage lambda competitor DNA (Promega, USA) and 5% (w/v) sucrose. Samples were resolved on a native PAGE gel and the percentage of dimer transfer for each time point was calculated from the intensity of the acceptor relative to the total Cy5 donor and acceptor fluorescence. The data was adjusted to give 0% transferred at time 0. All experiments were repeated at least three times using different nucleosome assemblies.

### REFERENCES

Anderson, J. D., Thastrom, A., and Widom, J. (2002). Spontaneous access of proteins to buried nucleosomal DNA target sites occurs via a mechanism that is distinct from nucleosome translocation. Mol Cell Biol 22, 7147-7157.
Bannister, A. J., Zegerman, P., Partridge, J. F., Miska, E. A., Thomas, J. O., Allshire, R. C., and Kouzarides, T. (2001). Selective recognition of methylated lysine 9 on histone H3 by the HP1 chromo domain. Nature 410, 120-124.
Bash, R., Wang, H., Anderson, C., Yodh, J., Hager, G., Lindsay, S. M., and Lohr, D. (2006). AFM imaging of protein movements: histone H2A-H2B release during nucleosome remodeling. FEBS Lett 580, 4757-4761.
Bruno, M., Flaus, A., Stockdale, C., Rencurel, C., Ferreira, H., and Owen-Hughes, T. (2003). Histone H2A/H2B dimer exchange by ATP-dependent chromatin remodeling activities. Mol Cell 12, 1599-1606.
Celic, I., Masumoto, H., Griffith, W. P., Meluh, P., Cotter, R. J., Boeke, J. D., and Verreault, A. (2006). The sirtuins hst3 and Hst4p preserve genome integrity by controlling histone h3 lysine 56 deacetylation. Curr Biol 16, 1280-1289.
Celic, I., Verreault, A., and Boeke, J. D. (2008). Histone H3 K56 hyperacetylation perturbs replisomes and causes DNA damage. Genetics 179, 1769-1784.
Chandy, M., Gutierrez, J. L., Prochasson, P., and Workman, J. L. (2006). SWI/SNF displaces SAGA-acetylated nucleosomes. Eukaryot Cell 5, 1738-1747.
Chen, C. C., Carson, J. J., Feser, J., Tamburini, B., Zabaronick, S., Linger, J., and Tyler, J. K. (2008). Acetylated lysine 56 on histone H3 drives chromatin assembly after repair and signals for the completion of repair. Cell 134, 231-243.
Cosgrove, M. S., Boeke, J. D., and Wolberger, C. (2004). Regulated nucleosome mobility and the histone code. Nat Struct Mol Biol 11, 1037-1043.
Dawson, P. E., Muir, T. W., Clark-Lewis, I., and Kent, S. B. (1994). Synthesis of proteins by native chemical ligation. Science 266, 776-779.
Dhalluin, C., Carlson, J. E., Zeng, L., He, C., Aggarwal, A. K., and Zhou, M. M. (1999). Structure and ligand of a histone acetyltransferase bromodomain. Nature 399, 491-496.
Dorigo, B., Schalch, T., Bystricky, K., and Richmond, T. J. (2003). Chromatin fiber folding: requirement for the histone H4N-terminal tail. J Mol Biol 327, 85-96.
Driscoll, R., Hudson, A., and Jackson, S. P. (2007). Yeast Rtt109 promotes genome stability by acetylating histone H3 on lysine 56. Science 315, 649-652.
Eggeling, C., Berger, S., Brand, L., Fries, J. R., Schaffer, J., Volkmer, A., and Seidel, C. A. (2001). Data registration and selective single-molecule analysis using multi-parameter fluorescence detection. J Biotechnol 86, 163-180.
Ferreira, H., Flaus, A., and Owen-Hughes, T. (2007). Histone modifications influence the action of Snf2 family remodelling enzymes by different mechanisms. J Mol Biol 374, 563-579.
Flaus, A., and Richmond, T. J. (1998). Positioning and stability of nucleosomes on MMTV 3′LTR sequences. J Mol Biol 275, 427-441.
Garcia, B. A., Hake, S. B., Diaz, R. L., Kauer, M., Morris, S. A., Recht, J., Shabanowitz, J., Mishra, N., Strahl, B. D., Allis, C. D., and Hunt, D. F. (2007). Organismal differences in post-translational modifications in histones H3 and H4. J Biol Chem 282, 7641-7655.
Grunstein, M. (1997). Histone acetylation in chromatin structure and transcription. Nature 389, 349-352.
Han, J., Zhou, H., Horazdovsky, B., Zhang, K., Xu, R. M., and Zhang, Z. (2007). Rtt109 acetylates histone H3 lysine 56 and functions in DNA replication. Science 315, 653-655.
Huynh, V. A., Robinson, P. J., and Rhodes, D. (2005). A method for the in vitro reconstitution of a defined “30 nm” chromatin fibre containing stoichiometric amounts of the linker histone. J Mol Biol 345, 957-968.
Hyland, E. M., Cosgrove, M. S., Molina, H., Wang, D., Pandey, A., Cottee, R. J., and Boeke, J. D. (2005). Insights into the role of histone H3 and histone H4 core modifiable residues in Saccharomyces cerevisiae. Mol Cell Biol 25, 10060-10070.
Jacobson, R. H., Ladurner, A. G., King, D. S., and Tjian, R. (2000). Structure and function of a human TAFII250 double bromodomain module. Science 288, 1422-1425.
Jenuwein, T., and Allis, C. D. (2001). Translating the histone code. Science 293, 1074-1080.
Kasten, M., Szerlong, H., Erdjument-Bromage, H., Tempst, P., Werner, M., and Cairns, B. R. (2004). Tandem bromodomains in the chromatin remodeler RSC recognize acetylated histone H3 Lys14. Embo J 23, 1348-1359.
Koopmans, W. J., Brehm, A., Logie, C., Schmidt, T., and van Noort, J. (2007). Single-pair FRET microscopy reveals mononucleosome dynamics. J Fluoresc 17, 785-795.
Kouzarides, T. (2007). Chromatin modifications and their function. Cell 128, 693-705.
Lachner, M., O'Carroll, D., Rea, S., Mechtler, K., and Jenuwein, T. (2001). Methylation of histone H3 lysine 9 creates a binding site for HP1 proteins. Nature 410, 116-120.
Li, G., and Widom, J. (2004). Nucleosomes facilitate their own invasion. Nat Struct Mol Biol 11, 763-769.
Li, H., Ilin, S., Wang, W., Duncan, E. M., Wysocka, J., Allis, C. D., and Patel, D. J. (2006). Molecular basis for site-specific read-out of histone H3K4me3 by the BPTF PHD finger of NURF. Nature 442, 91-95.
Li, Q., Zhou, H., Wurtele, H., Davies, B., Horazdovsky, B., Verreault, A., and Zhang, Z. (2008). Acetylation of histone H3 lysine 56 regulates replication-coupled nucleosome assembly. Cell 134, 244-255.
Lowary, P. T., and Widom, J. (1998). New DNA sequence rules for high affinity binding to histone octamer and sequence-directed nucleosome positioning. J Mol Biol 276, 19-42.
Luger, K., Mader, A. W., Richmond, R. K., Sargent, D. F., and Richmond, T. J. (1997). Crystal structure of the nucleosome core particle at 2.8 A resolution. Nature 389, 251-260.
Luger, K., Rechsteiner, T. J., and Richmond, T. J. (1999). Preparation of nucleosome core particle from recombinant histones. Methods Enzymol 304, 3-19.
Masumoto, H., Hawke, D., Kobayashi, R., and Verreault, A. (2005). A role for cell-cycle-regulated histone H3 lysine 56 acetylation in the DNA damage response. Nature 436, 294-298.
McGinty, R. K., Kim, J., Chatterjee, C., Roeder, R. G., and Muir, T. W. (2008). Chemically ubiquitylated histone H2B stimulates hDot1L-mediated intranucleosomal methylation. Nature 453, 812-816.
Neumann, H., Peak-Chew, S. Y., and Chin, J. W. (2008). Genetically encoding N(epsilon)-acetyllysine in recombinant proteins. Nat Chem Biol 4, 232-234.
Ozdemir, A., Spicuglia, S., Lasonder, E., Vermeulen, M., Campsteijn, C., Stunnenberg, H. G., and Logie, C. (2005). Characterization of lysine 56 of histone H3 as an acetylation site in Saccharomyces cerevisiae. J Biol Chem 280, 25949-25952.
Park, Y. J., Dyer, P. N., Tremethick, D. J., and Luger, K. (2004). A new fluorescence resonance energy transfer approach demonstrates that the histone variant H2AZ stabilizes the histone octamer within the nucleosome. J Biol Chem 279, 24274-24282.
Peterson, C. L., and Laniel, M. A. (2004). Histones and histone modifications. Curr Biol 14, R546-551.
Philo, J. S. (2006). Improved methods for fitting sedimentation coefficient distributions derived by time-derivative techniques. Anal Biochem 354, 238-246.
Rackham, O., and Chin, J. W. (2005). A network of orthogonal ribosome x mRNA pairs. Nat Chem Biol 1, 159-166.
Robinson, P. J., An, W., Routh, A., Martino, F., Chapman, L., Roeder, R. G., and Rhodes, D. (2008). 30 nm chromatin fibre decompaction requires both H4-K16 acetylation and linker histone eviction. J Mol Biol 381, 816-825.
Rufiange, A., Jacques, P. E., Bhat, W., Robert, F., and Nourani, A. (2007). Genome-wide replication-independent histone H3 exchange occurs predominantly at promoters and implicates H3 K56 acetylation and Asf1. Mol Cell 27, 393-405.
Ruthenburg, A. J., Li, H., Patel, D. J., and Allis, C. D. (2007). Multivalent engagement of chromatin modifications by linked binding modules. Nat Rev Mol Cell Biol 8, 983-994.
Saha, A., Wittmeyer, J., and Cairns, B. R. (2002). Chromatin remodeling by RSC involves ATP-dependent DNA translocation. Genes Dev 16, 2120-2134.
Shahbazian, M. D., and Grunstein, M. (2007). Functions of site-specific histone acetylation and deacetylation. Annu Rev Biochem 76, 75-100.
Shogren-Knaak, M., Ishii, H., Sun, J. M., Pazin, M. J., Davie, J. R., and Peterson, C. L. (2006). Histone H4-K16 acetylation controls chromatin structure and protein interactions. Science 311, 844-847.
Simon, M. D., Chu, F., Racki, L. R., de la Cruz, C. C., Burlingame, A. L., Panning, B., Narlikar, G. J., and Shokat, K. M. (2007). The site-specific installation of methyl-lysine analogs into recombinant histones. Cell 128, 1003-1012.
Stafford, W. F., 3rd (1992). Boundary analysis in sedimentation transport experiments: a procedure for obtaining sedimentation coefficient distributions using the time derivative of the concentration profile. Anal Biochem 203, 295-301.
Sterner, D. E., and Berger, S. L. (2000). Acetylation of histones and transcription-related factors. Microbiol Mol Biol Rev 64, 435-459.
Taverna, S. D., Li, H., Ruthenburg, A. J., Allis, C. D., and Patel, D. J. (2007a). How chromatin-binding modules interpret histone modifications: lessons from professional pocket pickers. Nat Struct Mol Biol 14, 1025-1040.
Taverna, S. D., Ueberheide, B. M., Liu, Y., Tackett, A. J., Diaz, R. L., Shabanowitz, J., Chait, B. T., Hunt, D. F., and Allis, C. D. (2007b). Long-distance combinatorial linkage between methylation and acetylation on histone H3N termini. Proc Natl Acad Sci USA 104, 2086-2091.
Wang, K., Neumann, H., Peak-Chew, S. Y., and Chin, J. W. (2007). Evolved orthogonal ribosomes enhance the efficiency of synthetic genetic code expansion. Nat Biotechnol 25, 770-777.
Williams, S. K., Truong, D., and Tyler, J. K. (2008). Acetylation in the globular core of histone H3 on lysine-56 promotes chromatin disassembly during transcriptional activation. Proc Natl Acad Sci USA 105, 9000-9005.
Wysocka, J., Swigut, T., Xiao, H., Milne, T. A., Kwon, S. Y., Landry, J., Kauer, M., Tackett, A. J., Chait, B. T., Badenhorst, P., et al. (2006). A PHD finger of NURF couples histone H3 lysine 4 trimethylation with chromatin remodelling. Nature 442, 86-90.
Xie, W., Song, C., Young, N. L., Sperling, A. S., Xu, F., Sridharan, R., Conway, A. E., Garcia, B. A., Plath, K., Clark, A. T., and Grunstein, M. (2009). Histone h3 lysine 56 acetylation is linked to the core transcriptional network in human embryonic stem cells. Mol Cell 33, 417-427.
Xu, F., Zhang, K., and Grunstein, M. (2005). Acetylation in histone H3 globular domain regulates gene expression in yeast. Cell 121, 375-385.
Xu, F., Zhang, Q., Zhang, K., Xie, W., and Grunstein, M. (2007). Sir2 deacetylates histone H3 lysine 56 to regulate telomeric heterochromatin structure in yeast. Mol Cell 27, 890-900.
Yang, B., Miller, A., and Kirchmaier, A. L. (2008). HST3/HST4-dependent deacetylation of lysine 56 of histone H3 in silent chromatin. Mol Biol Cell 19, 4993-5005.
Yang, X. J. (2004). Lysine acetylation and the bromodomain: a new partnership for signaling. Bioessays 26, 1076-1087.

### FIGURE LEGENDS

FIG. 1. Selection of an improved acetyl-lysyl tRNA synthetase/tRNA pair for the incorporation of acetyl-lysine in recombinant proteins. A. The active site of M. mazei PylRS bound to pyrrolysine (figure created using Pymol (www.pymol.org) and pdb file 2Q7H). The residues mutated relative to the wild-type sequence are shown as sticks. Residues in cyan are mutated in the progenitor AcKRS-1 and were randomized again in the new library, A267 (magenta) was only included in the new library. B. Characterization of a more efficient acetyl-lysyl tRNA synthetase/tRNA_{CUA} pair. Myoglobin-His₆ was expressed in E. coli DH 10B from pMyo4TAG PylT (Neumann et al., 2008) (containing a hexa-histidine tagged myoglobin gene with an amber codon at position 4 and the gene encoding MbtRNA_{CUA}) in the presence or absence of 10 mM acetyl-lysine using either pBK AcKRS-1 or pBK AcKRS-3. The proteins were purified by Ni²⁺ chromatography and analysed by 4-12% SDS-PAGE or detected in total lysates by Western blot with an anti-His₆ antibody.

FIG. 2. The expression and purification of site-specifically acetylated histones and the assembly of histone octamers and nucleosomes. A. Schematic illustration showing the recombinant expression of site-specifically acetylated recombinant histones in E. coli and their reconstitution into histone octamers and nucleosomes. B. (Left) The expression, purification and TEV cleavage of histone H3 K14Ac is followed by SDS PAGE, (Right) Purified and TEV cleaved site specifically acetylated histones. (1) molecular weight marker, (2) H3 WT, (3) H3 K14Ac, (4) H3 K23Ac, (5) H3 K27Ac, (6) H3 K56Ac, (7) H2A WT, (8) H2A K9Ac, (9) H2B WT, (10) H2B K5Ac, (11) H2B K20Ac. C. Electrospray ionization mass spectrometry demonstrates that the protein is homogeneously acetylated and MS/MS of tryptic peptides identifies the site of acetylation is at lysine 56, as genetically encoded. The smaller peak to the right of the main peak is 98 Da heavier and corresponds to a phosphate from buffer associated with the histone. This peak can be removed by further desalting of the histone with a ZIP-tip. D. H3 K56Ac assembles into octamers with a comparable efficiency to unmodified H3. Denaturing (4-12%) SDS-PAGE of assembled octamers. The acetylation of H3 in the octamer is confirmed by Western blot with an anti-acetyl-lysine antibody. E. Reconstitution of unmodified octamers and octamers bearing H3 K56 acetylation into nucleosomes with 197 bp 601 DNA, nucleosomes, and octamers were resolved by 0.8% agarose gel and stained with ethidium bromide.

FIG. 3. Nucleosomal stability and dynamic partial unwrapping of nucleosomal DNA measured by FRET using three-way labelled nucleosomes. A Schematic of the nucleosome highlighting the locations of the fluorescence donor Cy3 (green) at the 5′ end of the DNA, the acceptor dye Cy5 (red) coupled to histone H2A K119C, and the site of acetylation, H3 K56Ac (blue). The figure was created using the pdb file 1KX5 and pymol (www.pymol.org). B. Analysis of nucleosome reconstitution by 0.8% agarose gel electrophoresis, where lane 1=100 bp DNA ladder, lane 2=naked Cy3-labelled DNA, lane 3=Cy5-labelled H2A K119C nucleosome reconstitution with wild-type H3, lane 4=Cy5-labelled H2A K119C nucleosome reconstitution with H3 K56Ac. C. The salt-induced dissociation of nucleosome core particles can be monitored by FRET. (Left) Increasing [NaCl] from 0 (red) to 1.75 M (violet) leads to decreased FRET emission from Cy5 and increased Cy3 emission (arrows). Excitation wavelength was set at 515 nm. (Right) Equilibrium dissociation curves were obtained by monitoring changes in fluorescence donor and acceptor emission at 565 and 670 nm, respectively. Data were normalized using the upper and lower plateau values as baselines, with wild-type nucleosomes in orange and H3 K56Ac nucleosomes in magenta.

FIG. 4. spFRET experiments on transient unwrapping of DNA and DNA breathing demonstrate that K56 acetylation promotes local unwrapping near the entry exit points of the nucleosome. A. Schematic of the labelling positions on the nucleosome DNA. The end-label fluorophore pair (Cy3/Atto647N) is close to the entry exit point of the nucleosome at position −6 and the internal-label pair is at −29 from the entry exit point. The position of K56 is shown in blue. The figure was created using the pdb file 1KX5 and pymol (www.pymol.org). B&C. spFRET efficiency measured for nucleosomes reconstituted with internally- or end-labelled DNA, respectively, using a combination of native PAGE, ALEX and FCS as described in the experimental section.

FIG. 5. Assembly and sedimentation analysis of nucleosome arrays bearing homogeneously acetylated nucleosomes. A. Titration of purified histone H3 K56Ac octamers to assemble chromatin arrays containing 61 repeats of 197 bp of the 601 nucleosome positioning sequence. A retarded gel shift indicates loading of the DNA array with histone octamers. Excess histone octamer forms nucleosome core particles (NCPs) with competitor DNA (crDNA). Conditions of lane 4 were used to reconstitute DNA arrays in subsequent experiments. B. DNA arrays were reconstituted with saturating amounts of histone octamer and with increasing amounts of H5 linker histone in order to induce compaction. Chromatin arrays were folded in 1 mM MgCl₂, 20 mM TEA pH 7.4 and the degree of the compaction was measured quantitatively by sedimentation velocity analysis.

FIG. 6. A&B H3 K56 acetylated nucleosomes cause minimal alteration to the initial rate of RSC or SWI/SNF repositioning. Competitive repositioning assays were performed using 1 pmol each of H3 K56 acetylated and wild-type nucleosomes, 1 mM ATP and 41 fmol of RSC (A) or 115 fmol of SWI/SNF (B). A representative native PAGE gel of the repositioning assay is shown for each remodeller. The initial rate estimate for repositioning of H3 K56Ac nucleosomes relative to wild-type for RSC was 1.2 fold±0.1 (mean±standard error of the mean) and for SWI/SNF 1.4 fold±0.2. Each experiment was repeated in triplicate. * indicates the P position. WT, wild-type. C&D H3 K56Ac and wild-type nucleosomes exhibit equivalent remodeller driven dimer transfer. Remodeller dimer transfer was performed using 0.25 pmol of donor nucleosomes assembled with Cy5 labelled H2A onto 54A18 DNA fragments, 0.75 pmol of wild-type tetrasome acceptor assembled on 0W0 DNA fragments, 1 mM ATP and 83 fmol RSC(C) or 230 fmol SWI/SNF (D). For each dimer transfer experiment, a representative Cy5 scan of the native PAGE gel is shown. Both RSC and SWI/SNF caused a 1.2 fold increase of the percentage of dimer transfer for H3 K56Ac nucleosomes relative to wild-type at the finish of their respective time courses. As the standard error of the mean was large in both cases, 0.1 and 0.2 for RSC and SWI/SNF, respectively, there was no significant change in the percentage of dimer transfer. Each experiment was repeated in triplicate.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A tRNA synthetase capable of binding N^{ε}-acetyl lysine, wherein said synthetase comprises a polypeptide having at least 90% sequence identity to the amino acid sequence of MbPylRS, and wherein said synthetase comprises a L266M mutation.

2. A tRNA synthetase according to claim 1 wherein said tRNA synthetase comprises amino acid sequence corresponding to the amino acid sequence of at least L266 to C313 of MbPylRS, or a sequence having at least 90% identity thereto.

3. A tRNA synthetase according to claim 1 wherein said polypeptide comprises a mutation relative to the wild type MbPylRS sequence at one or more of L270, Y271, L274 or C313.

4. A tRNA synthetase according to claim 3 wherein said at least one mutation is at L270, L274 or C313.

5. A tRNA synthetase according to claim 1 which comprises Y271F.

6. A tRNA synthetase according to claim 1 which comprises L2701, Y271F, L274A, and C313F.

7. A nucleic acid comprising nucleotide sequence encoding a polypeptide according to claim 1.

8-9. (canceled)

10. A method of making a polypeptide comprising N^{ε}-acetyl lysine comprising arranging for the translation of a RNA encoding said polypeptide, wherein said RNA comprises an amber codon, wherein said translation is carried out in the presence of a polypeptide according to claim 1 and in the presence of tRNA which recognises the amber codon and is capable of being charged with N^{ε}-acetyl lysine, and in the presence of N^{ε}-acetyl lysine.

11. A method according to claim 10 wherein said translation is carried out in the presence of an inhibitor of deacetylation.

12. A method according to claim 11 wherein said inhibitor comprises nicotinamide (NAM).

13. A method according to claim 10 wherein said polypeptide comprises a histone protein.

14. The method according to claim 13, wherein the histone comprises a histone selected from H2A, H2B and H3.

15. The method according to claim 14, wherein either
(a) the histone is H3 and the lysine residue is lysine 56; or
(b) the histone is H2A and the lysine residue is lysine 9; or
(c) the histone is H2B and the lysine residue is lysine 5 and/or lysine 20.

16. (canceled)

17. A homogenous recombinant histone, wherein said protein is made by a method according to claim 10.

18. A vector comprising nucleic acid according to claim 7.

19. A vector according to claim 18, said vector further comprising nucleic acid sequence encoding a tRNA substrate of said tRNA synthetase.

20. A vector according to claim 19 wherein said tRNA substrate is encoded by the MbPylT gene.

21. A cell comprising a nucleic acid according to claim 7.

22. A cell according to claim 21 which further comprises an inactivated de-acetylase gene.

23. A cell according to claim 22 wherein said deactivated de-acetylase gene comprises a deletion or disruption of CobB.

24. A kit comprising a vector according to claim 21 and an amount of nicotinamide.

25. (canceled)

26. A cell comprising a vector according to claim 18.

27. A cell according to claim 26 which further comprises an inactivated de-acetylase gene.

28. A cell according to claim 27 wherein said deactivated de-acetylase gene comprises a deletion or disruption of CobB.

29. A kit comprising a cell according to claim 21 and an amount of nicotinamide.
